(19) United States
(12) Patent Application Publication
(10) Pub. No.: US 2023/0366017 A1
(43) Pub. Date: Nov. 16, 2023

(54) Methods and Compositions for Targeted Single Cell cDNA Sequencing

(71) Applicant: National University of Singapore, Singapore (SG)

(72) Inventors: Jonathan Adam Scolnick, Singapore (SG); Hui Qi Hong, Singapore (SG)

(21) Appl. No.: 18/025,510

(22) PCT Filed: Sep. 9, 2021
(86) PCT No.: PCT/IB2021/058218

§ 371 Date: Mar. 9, 2023

Related U.S. Application Data
(60) Provisional application No. 63/076,672, filed on Sep. 10, 2020.

(51) Int. Cl. C12Q 1/6869

(52) U.S. Cl. CPC C12Q 1/6869

(57) ABSTRACT

The present invention generally provides, in various embodiments, methods of generating tagged DNA amplicons for sequencing. In one embodiment, the method comprises a) annealing a first oligonucleotide to a nucleic acid template comprising a target nucleic acid molecule sequence and a tag sequence, wherein the target nucleic acid molecule sequence comprises a locus of interest; b) performing nucleic acid template-directed nucleic acid extension from the annealed first oligonucleotide to provide a first extension product; c) circularizing the first extension product to produce a circularized DNA template; and d) performing circularized DNA template-directed nucleic acid amplification to produce a tagged DNA amplicon for sequencing. The methods are useful for linking a distant locus-of-interest to a reverse transcription primer.

### RELATED APPLICATION(S)

This application claims the benefit of U.S. Provisional Application No. 63/076,672, filed on Sep. 10, 2020. The entire teachings of the above application are incorporated herein by reference.

### INCORPORATION BY REFERENCE OF MATERIAL IN ASCII TEXT FILE

This application incorporates by reference the Sequence Listing contained in the following ASCII text file being submitted concurrently herewith:
a) File name: 44591155001_SEQUENCELISTING.txt; created Sep. 9, 2021, 12,288 bytes in size.

### BACKGROUND

The ability to obtain single-cell transcriptomic information from an admixed cell population is important for both molecular biology research and medical applications. A sensitive and rapid method for generating tagged DNA amplicons for sequencing a locus of interest that is distant from transcript ends is needed.

### SUMMARY

The present invention provides methods of generating tagged DNA amplicons for sequencing.

In one aspect, the invention provides a method of generating a tagged DNA amplicon for sequencing, comprising the steps of:
a) annealing a first oligonucleotide to a nucleic acid template comprising a target nucleic acid molecule sequence, a tag sequence and at least a portion of a first universal sequence, wherein the target nucleic acid molecule sequence comprises a locus of interest;
b) performing nucleic acid template-directed nucleic acid extension from the annealed first oligonucleotide to produce a first extension product comprising all or a portion of the target nucleic acid molecule sequence that comprises the locus of interest, the tag sequence and at least a portion of the first universal sequence;
c) circularizing the first extension product to produce a circularized DNA template comprising the locus of interest, the tag sequence, at least a portion of the first universal sequence and at least a portion of a second universal sequence; and
d) performing circularized DNA template-directed nucleic acid amplification to produce a DNA amplicon comprising the locus of interest, the tag sequence, at least a portion of the first universal sequence and at least a portion of the second universal sequence,
   thereby generating the tagged DNA amplicon for sequencing.

In some embodiments, the method further comprises performing first extension product-directed nucleic acid amplification to amplify the first extension product. In some embodiments, the first extension product-directed nucleic acid amplification is performed using:
a) the first oligonucleotide; and
b) a first primer comprising at least a portion of the first universal sequence.

In another aspect, the invention provides a method of generating a tagged DNA amplicon for sequencing, comprising the steps of:
a) providing a nucleic acid template comprising a target nucleic acid molecule sequence, a tag sequence and at least a portion of a first universal sequence, wherein the target nucleic acid molecule sequence comprises a locus of interest;
b) performing nucleic acid template-directed nucleic acid amplification to produce a first amplification product by:
   i. contacting the nucleic acid template with a reaction mixture comprising a first oligonucleotide and a first primer under conditions in which the primers anneal to complementary nucleotide sequences in the nucleic acid template, wherein the first primer comprises at least a portion of the first universal sequence; and
   ii. extending each of the first oligonucleotide and the first primer;
c) circularizing the first amplification product to produce a circularized DNA template comprising the locus of interest, the tag sequence, at least a portion of the first universal sequence and at least a portion of a second universal sequence; and
d) performing circularized DNA template-directed nucleic acid amplification to produce a DNA amplicon comprising the locus of interest, the tag sequence, at least a portion of the first universal sequence and at least a portion of the second universal sequence,
   thereby generating the tagged DNA amplicon for sequencing.

In another aspect, the invention provides a method of generating a tagged DNA amplicon for sequencing, comprising the steps of:
a) providing a nucleic acid template comprising, from the 5′ end to the 3′ end, a truncated first universal sequence, a tag sequence, a target nucleic acid molecule sequence and a first template switching oligo (TSO1) sequence, wherein the target nucleic acid molecule sequence comprises a locus of interest;
b) performing nucleic acid template-directed nucleic acid amplification to produce a first amplification product by:
   i. contacting the nucleic acid template with a reaction mixture comprising a first oligonucleotide and a first reverse primer under conditions in which the first oligonucleotide and the first reverse primer anneal to complementary nucleotide sequences in the nucleic acid template, wherein:
      1) the first oligonucleotide comprises, from the 5′ end to the 3′ end, a second universal sequence and a target nucleic acid molecule-specific sequence; and
      2) the first reverse primer comprises, from the 5′ end to the 3′ end, a second universal sequence, [i7], the missing first universal sequence and at least a portion of the truncated first universal sequence; and
   ii. extending each of the first oligonucleotide and the first reverse primer;
c) circularizing the first amplification product to produce a circularized DNA template comprising the second universal sequence, the locus of interest, the tag sequence, the first universal sequence and [i7]; and
d) performing circularized DNA template-directed nucleic acid amplification to produce a DNA amplicon comprising, from 5′ end to the 3′ end, the Illumina P5 sequence, the locus of interest, the second universal sequence, [i7], the first universal sequence, the tag sequence and the Illumina P7 sequence,
   thereby generating the tagged DNA amplicon for sequencing.

In another aspect, the invention provides a method of generating a tagged DNA amplicon for sequencing, comprising the steps of:
a) providing a nucleic acid template comprising, from the 5′ end to the 3′ end, a truncated first universal sequence, a tag sequence, a second template switching oligo (TSO2) sequence and a target nucleic acid molecule sequence, wherein the target nucleic acid molecule sequence comprises a locus of interest;
b) performing nucleic acid template-directed nucleic acid amplification to produce a first amplification product by:
   i. contacting the nucleic acid template with a reaction mixture comprising a first oligonucleotide and a first reverse primer under conditions in which the first oligonucleotide and the first reverse primer anneal to complementary nucleotide sequences in the nucleic acid template, wherein:
      1) the first oligonucleotide comprises, from the 5′ end to the 3′ end, a second universal sequence and a target nucleic acid molecule-specific sequence; and
      2) the first reverse primer comprises, from the 5′ end to the 3′ end, a second universal sequence, [i7], the missing first universal sequence and at least a portion of the truncated first universal sequence; and
   ii. extending each of the first oligonucleotide and the first reverse primer;
c) circularizing the first amplification product to produce a circularized DNA template comprising the second universal sequence, the locus of interest, the TSO2 sequence, the tag sequence, the first universal sequence and [i7]; and
d) performing circularized DNA template-directed nucleic acid amplification to produce a DNA amplicon comprising, from 5′ end to the 3′ end, the Illumina P5 sequence, the locus of interest, the second universal sequence, [i7], the first universal sequence, the TSO2 sequence, the tag sequence and the Illumina P7 sequence,
   thereby generating the tagged DNA amplicon for sequencing.

In some embodiments, the nucleic acid template is a complementary DNA (cDNA) template. In some embodiments, the cDNA template is obtained by reverse transcribing an RNA from a single cell. In some embodiments, the RNA is mRNA. In some embodiments, the nucleic acid template is a double-stranded DNA molecule (e.g., an amplicon) produced by amplification of a cDNA molecule.

In some embodiments, the cDNA template corresponds to a first strand cDNA that comprises, from the 5′ end to the 3′ end, at least a portion of a first universal sequence, the tag sequence, the target nucleic acid molecule sequence and a first template switching oligo (TSO1) sequence. In other embodiments, the cDNA template corresponds to a second strand cDNA that comprises, from the 5′ end to the 3′ end, at least a portion of a first universal sequence, the tag sequence, a second template switching oligo (TSO2) sequence and the target nucleic acid molecule sequence.

In some embodiments, the locus of interest comprises a mutation, a polymorphism, an insertion, a deletion, a gene fusion, an edited nucleotide, a modified nucleotide, a transgene or a combination thereof.

In some embodiments, the tag sequence comprises a cell identification tag or a unique molecular identifier (UMI) sequence, or a combination thereof.

In some embodiments, the method comprises a single circularizing step.

In some embodiments, circularizing the first extension product comprises an intramolecular ligation mediated by Gibson assembly.

In some embodiments, the method is used to make a sequencing library.

In another aspect, the invention provides a tagged DNA amplicon for sequencing, comprising, from the 5′ end to the 3′ end, a locus of interest, at least a portion of a second universal sequence, at least a portion of a first universal sequence and a tag sequence. In some embodiments, the tagged DNA amplicon comprises, from the 5′ end to the 3′ end, the locus of interest, the second universal sequence, the first universal sequence and the tag sequence.

In some embodiments, the tagged DNA amplicon comprises, from the 5′ end to the 3′ end, an Illumina P5 sequence, the locus of interest, the second universal sequence, the first universal sequence, the tag sequence and the Illumina P7 sequence.

In some embodiments, the tagged DNA amplicon comprises, from the 5′ end to the 3′ end, an Illumina P5 sequence, the locus of interest, the second universal sequence, [i7], the first universal sequence, the tag sequence and the Illumina P7 sequence.

In some embodiments, the tagged DNA amplicon comprises, from the 5′ end to the 3′ end, an Illumina P5 sequence, the locus of interest, the second universal sequence, [i7], the first universal sequence, the tag sequence, a poly(T) sequence and the Illumina P7 sequence.

In some embodiments, the tagged DNA amplicon comprises, from the 5′ end to the 3′ end, an Illumina P5 sequence, the locus of interest, the second universal sequence, [i7], a second template switching oligo (TSO2) sequence and the Illumina P7 sequence.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIGS. 1A and 1B depict non-limiting examples of molecular design of SIT-seq (e.g., for a cDNA generated from the 10× Genomics 3′ Gene expression kit or a 3′ biased RNA-Seq library). The molecular design links a distant locus of interest to the reverse transcription primer (shown as the 10× Genomics cell ID sequence). FIG. 1C depicts a Sanger sequencing chromatogram of the Illumina library sequencing with P5 primer. Read 1 and Read 2 represent the first and the second universal sequence, respectively.

FIG. 2 depicts a non-limiting example of molecular design of SIT-seq (e.g., for a cDNA generated from the 10× Genomics 5′ Gene expression kit or a 5′ biased RNA-Seq library).

FIG. 3 depicts a non-limiting example of sequencing of SIT-seq library on Illumina platforms.

FIG. 4 depicts a non-limiting example of target enrichment using hybridization and extension.

FIG. 5 depicts a non-limiting example of target enrichment using RNase H-dependent PCR (rhPCR).

### DETAILED DESCRIPTION

A description of example embodiments follows.

Unless defined otherwise, all technical and scientific terms used herein have the same meaning as commonly understood by one of ordinary skill in the art to which this invention pertains.

In one aspect, the invention provides a method of generating a tagged DNA amplicon for sequencing, comprising the steps of:
a) annealing a first oligonucleotide to a nucleic acid template comprising a target nucleic acid molecule sequence, a tag sequence and at least a portion of a first universal sequence, wherein the target nucleic acid molecule sequence comprises a locus of interest;
b) performing nucleic acid template-directed nucleic acid extension from the annealed first oligonucleotide to produce a first extension product comprising all or a portion of the target nucleic acid molecule sequence that comprises the locus of interest, the tag sequence and at least a portion of the first universal sequence;
c) circularizing the first extension product to produce a circularized DNA template comprising the locus of interest, the tag sequence, at least a portion of the first universal sequence and at least a portion of a second universal sequence; and
d) performing circularized DNA template-directed nucleic acid amplification to produce a DNA amplicon comprising the locus of interest, the tag sequence, at least a portion of the first universal sequence and at least a portion of the second universal sequence,
   thereby generating the tagged DNA amplicon for sequencing.

In some embodiments:
a) the nucleic acid template comprises a truncated first universal sequence;
b) the circularized DNA template comprises the entire first universal sequence and the entire second universal sequence; and
c) the DNA amplicon comprises the entire first universal sequence and the entire second universal sequence.

In some embodiments, the method further comprises performing first extension product-directed nucleic acid amplification to amplify the first extension product.

In some embodiments, the first extension product-directed nucleic acid amplification is performed using:
a) the first oligonucleotide; and
b) a first reverse primer comprising at least a portion of the truncated first universal sequence.

In another aspect, the invention provides a method of generating a tagged DNA amplicon for sequencing, comprising the steps of:
a) providing a nucleic acid template comprising a target nucleic acid molecule sequence, a tag sequence and at least a portion of a first universal sequence, wherein the target nucleic acid molecule sequence comprises a locus of interest;
b) performing nucleic acid template-directed nucleic acid amplification to produce a first amplification product by:
   i. contacting the nucleic acid template with a reaction mixture comprising a first oligonucleotide and a first primer under conditions in which the first oligonucleotide and the first primer anneal to complementary nucleotide sequences in the nucleic acid template, wherein the first primer comprises at least a portion of the first universal sequence, and wherein the first oligonucleotide, the first primer or both comprise a second universal sequence; and
   ii. extending each of the first oligonucleotide and the first primer;
c) circularizing the first amplification product to produce a circularized DNA template comprising the locus of interest, the tag sequence, at least a portion of the first universal sequence and at least a portion of the second universal sequence; and
d) performing circularized DNA template-directed nucleic acid amplification to produce a DNA amplicon comprising the locus of interest, the tag sequence, at least a portion of the first universal sequence and at least a portion of the second universal sequence,
   thereby generating the tagged DNA amplicon for sequencing.

In another aspect, the invention provides a method of generating a tagged DNA amplicon for sequencing, comprising the steps of:
a) providing a nucleic acid template comprising, from the 5′ end to the 3′ end, a truncated first universal sequence, a tag sequence, a target nucleic acid molecule sequence and a first template switching oligo (TSO1) sequence, wherein the target nucleic acid molecule sequence comprises a locus of interest;
b) performing nucleic acid template-directed nucleic acid amplification to produce a first amplification product by:
   i. contacting the nucleic acid template with a reaction mixture comprising a first oligonucleotide and a first reverse primer under conditions in which the first oligonucleotide and the first reverse primer anneal to complementary nucleotide sequences in the nucleic acid template, wherein:
      1) the first oligonucleotide comprises, from the 5′ end to the 3′ end, a second universal sequence and a target nucleic acid molecule-specific sequence; and
      2) the first reverse primer comprises, from the 5′ end to the 3′ end, a second universal sequence, [i7], the missing first universal sequence and at least a portion of the truncated first universal sequence; and
   ii. extending each of the first oligonucleotide and the first reverse primer;
c) circularizing the first amplification product to produce a circularized DNA template comprising the second universal sequence, the locus of interest, the tag sequence, the first universal sequence and [i7]; and
d) performing circularized DNA template-directed nucleic acid amplification to produce a DNA amplicon comprising, from 5′ end to the 3′ end, the Illumina P5 sequence, the locus of interest, the second universal sequence, [i7], the first universal sequence, the tag sequence and the Illumina P7 sequence,
   thereby generating the tagged DNA amplicon for sequencing.

In another aspect, the invention provides a method of generating a tagged DNA amplicon for sequencing, comprising the steps of:
a) providing a nucleic acid template comprising, from the 5′ end to the 3′ end, a truncated first universal sequence, a tag sequence, a second template switching oligo (TSO2) sequence and a target nucleic acid molecule sequence, wherein the target nucleic acid molecule sequence comprises a locus of interest;
b) performing nucleic acid template-directed nucleic acid amplification to produce a first amplification product by:
   i. contacting the nucleic acid template with a reaction mixture comprising a first oligonucleotide and a first reverse primer under conditions in which the first oligonucleotide and the first reverse primer anneal to complementary nucleotide sequences in the nucleic acid template, wherein:
      1) the first oligonucleotide comprises, from the 5′ end to the 3′ end, a second universal sequence and a target nucleic acid molecule-specific sequence; and
      2) the first reverse primer comprises, from the 5′ end to the 3′ end, a second universal sequence, [i7], the missing first universal sequence and at least a portion of the truncated first universal sequence; and
   ii. extending each of the first oligonucleotide and the first reverse primer;
c) circularizing the first amplification product to produce a circularized DNA template comprising the second universal sequence, the locus of interest, the TSO2 sequence, the tag sequence, the first universal sequence and [i7]; and
d) performing circularized DNA template-directed nucleic acid amplification to produce a DNA amplicon comprising, from 5′ end to the 3′ end, the Illumina P5 sequence, the locus of interest, the second universal sequence, [i7], the first universal sequence, the TSO2 sequence, the tag sequence and the Illumina P7 sequence,
   thereby generating the tagged DNA amplicon for sequencing.

In some embodiments:
a) the nucleic acid template comprises a truncated first universal sequence;
b) the circularized DNA template comprises the entire first universal sequence and the entire second universal sequence; and
c) the DNA amplicon comprises the entire first universal sequence and the entire second universal sequence.

It should be noted that throughout this specification the term “comprising” is used to denote that embodiments of the invention “comprise” the noted features and as such, may also include other features. However, in the context of this invention, the term “comprising” may also encompass embodiments in which the invention “consists essentially of” the relevant features or “consists of” the relevant features.

The term “nucleotide” refers to naturally occurring ribonucleotide or deoxyribonucleotide monomers, as well as non-naturally occurring derivatives and analogs thereof. Accordingly, nucleotides can include, for example, nucleotides comprising naturally occurring bases (e.g., A, G, C, or T), nucleotides comprising modified bases (e.g., 7-deazaguanosine, or inosine) and nucleotides comprising modified ribose (e.g., locked nucleic acid (LNA)).

### Nucleic Acid Template

In some embodiments, the nucleic acid template is a complementary DNA (cDNA) template. As used herein, “complementary DNA” or “cDNA” refers to a nucleic acid molecule synthesized from a single-stranded RNA (e.g., messenger RNA (mRNA), non-polyadenylated RNA, microRNA) template in a reaction catalyzed by a reverse transcriptase enzyme. In some embodiments, the reaction catalyzed by the reverse transcriptase enzyme uses a RT primer selected from the group consisting of an oligo(dT) primer, a gene-specific primer, and a random oligomer. In some embodiments, the random oligomer is a random hexamer.

In some embodiments, the nucleic acid template is a double-stranded DNA molecule (e.g., an amplicon) produced by amplification of a cDNA molecule.

In some embodiments, the cDNA template is obtained by reverse transcribing a RNA (e.g., an mRNA) from a single cell. In other embodiments, the cDNA template is obtained by reverse transcribing a RNA (e.g., an mRNA) from a plurality of cells. Non-limiting examples of cells include mammalian cells, plant cells, bacterial cells and fungal cells. In some embodiments, the cell is a mammalian cell. In some embodiments, the cell is a cancer cell. In some embodiments, the cancer is blood cancer (leukemia, lymphoma or myeloma). In some embodiments, the cell is a metastasized cancer cell.

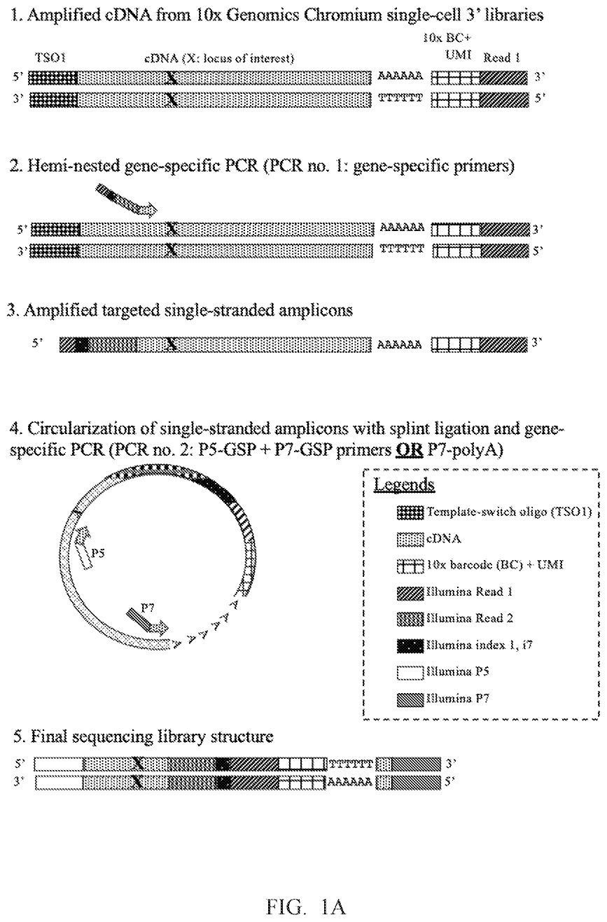

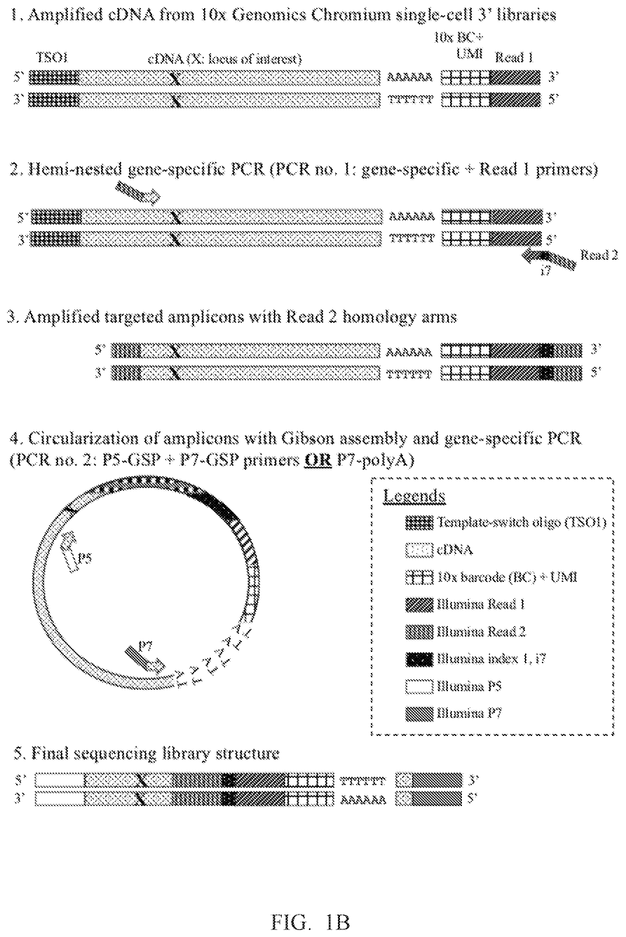

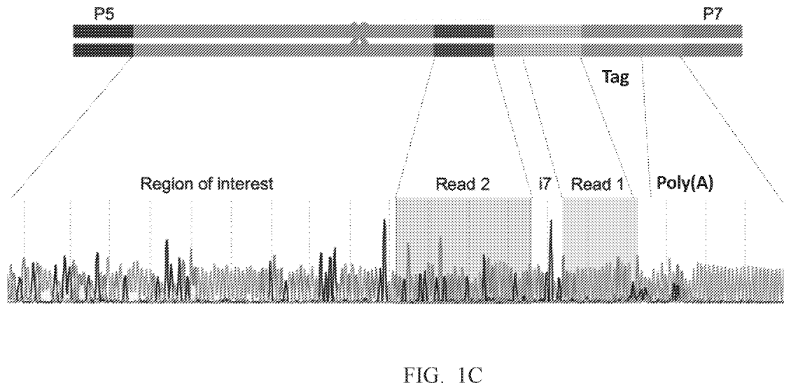

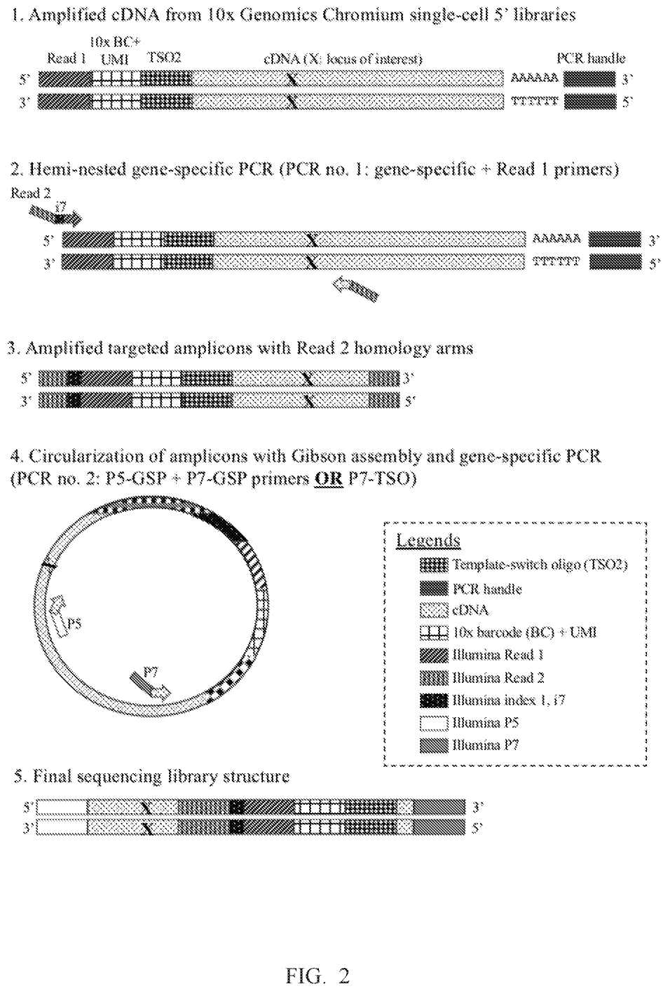

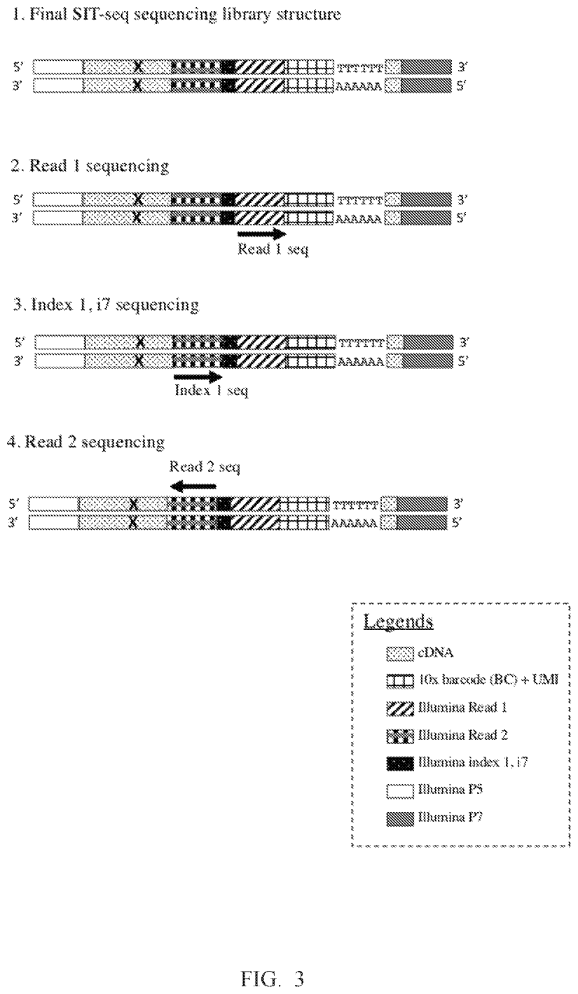

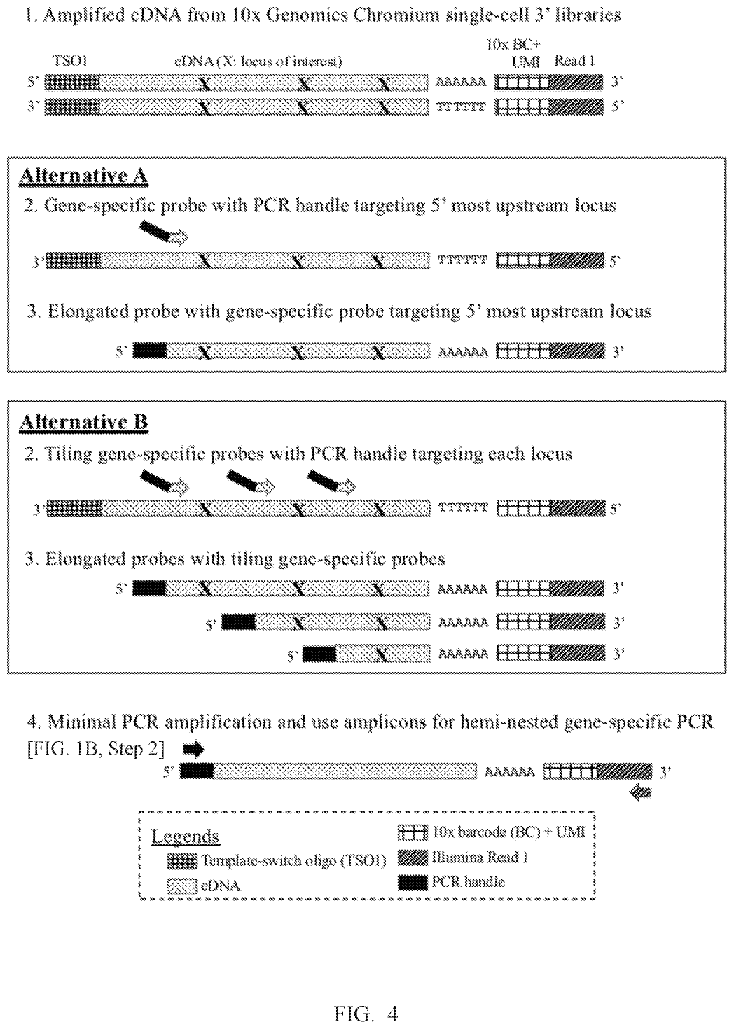

In some embodiments, the cDNA template corresponds to a cDNA of a 10× Genomics' 3′ RNA-Seq library (FIGS. 1A and 1B). In some embodiments, the cDNA template corresponds to a first strand cDNA. In some embodiments, the cDNA template comprises, from the 5′ end to the 3′ end, at least a portion of a first universal sequence, a tag sequence and a target nucleic acid molecule sequence. In some embodiments, the cDNA template comprises, from the 5′ end to the 3′ end, at least a portion of a first universal sequence, a tag sequence, a target nucleic acid molecule sequence and a first template switching oligo (TSO1) sequence. In some embodiments, the cDNA template comprises, from the 5′ end to the 3′ end, at least a portion of a first universal sequence, a tag sequence, a poly(T) sequence, and a target nucleic acid molecule sequence. In some embodiments, the cDNA template comprises, from the 5′ end to the 3′ end, at least a portion of a first universal sequence, a tag sequence, a poly(T) sequence, a target nucleic acid molecule sequence and a TSO1 sequence. In some embodiments, at least a portion of a first universal sequence is a truncated first universal sequence. In some embodiments, at least a portion of a first universal sequence is the entire first universal sequence.

In some embodiments, the TSO1 sequence comprises 5′ AAGCAGTGGTATCAACGCAGAGTACATrGrGrG 3′ (SEQ ID NO: 1). As used herein, “rG” represents riboguanosine.

In some embodiments, the TSO1 sequence comprises 5′ AGAGACAGATTGCGCAATG rGrGrG 3′ (SEQ ID NO: 2), wherein rG is riboguanosine.

In some embodiments, the TSO1 sequence comprises 5′ AAGCAGTGGTATCAACGCAGAGTACATrGrG+G 3′ (SEQ ID NO: 3), wherein rG is riboguanosine, and +G is a locked nucleic acid (LNA)-modified guanosine.

In some embodiments, the cDNA template corresponds to a cDNA of a 10× Genomics' 5′ RNA-Seq library (FIG. 2). In some embodiments, the cDNA template corresponds to a second strand cDNA. In some embodiments, the cDNA template comprises, from the 5′ end to the 3′ end, at least a portion of a first universal sequence, a tag sequence and a target nucleic acid molecule sequence. In some embodiments, the cDNA template comprises, from the 5′ end to the 3′ end, at least a portion of a first universal sequence, a tag sequence, a target nucleic acid molecule sequence and a poly(A) sequence. In some embodiments, the cDNA template comprises, from the 5′ end to the 3′ end, at least a portion of a first universal sequence, a tag sequence, a target nucleic acid molecule sequence and a PCR handle sequence. In some embodiments, the cDNA template comprises, from the 5′ end to the 3′ end, at least a portion of a first universal sequence, a tag sequence, a target nucleic acid molecule sequence, a poly(A) sequence and a PCR handle sequence. In some embodiments, at least a portion of a first universal sequence is a truncated first universal sequence. In some embodiments, at least a portion of a first universal sequence is the entire first universal sequence.

In some embodiments, the cDNA template comprises, from the 5′ end to the 3′ end, a second template switching oligo (TSO2) sequence and a target nucleic acid molecule sequence. In some embodiments, the cDNA template comprises, from the 5′ end to the 3′ end, a TSO2 sequence, a target nucleic acid molecule sequence and a poly(A) sequence. In some embodiments, the cDNA template comprises, from the 5′ end to the 3′ end, a TSO2 sequence, a target nucleic acid molecule sequence and a PCR handle sequence. In some embodiments, the cDNA template comprises, from the 5′ end to the 3′ end, a TSO2 sequence, a target nucleic acid molecule sequence, a poly(A) sequence and a PCR handle sequence.

In some embodiments, the PCR handle sequence comprises 5′ AAGCAGTGGTATCAACGCAGAGTAC 3′ (SEQ ID NO: 4).

In some embodiments, the TSO2 sequence comprises, from the 5′ end to the 3′ end, a truncated first universal sequence, the tag sequence, and 5′ TTTCTTATATrGrGrG 3′ (SEQ ID NO: 6). In some embodiments, the TSO2 sequence comprises, from the 5′ end to the 3′ end, 5′ CTACACGACGCTCTTCCGATCT 3′ (SEQ ID NO: 5), the tag sequence, and 5′ TTTCTTATATrGrGrG 3′ (SEQ ID NO: 6).

In some embodiments, the cDNA template is within a plurality of cDNAs.

In some embodiments, the method further comprises performing reverse transcription of the target nucleic acid molecule (e.g., target mRNA) to generate the nucleic acid template (e.g., cDNA template). In some embodiments, performing reverse transcription of the target nucleic acid molecule comprises contacting an mRNA (e.g., from a single cell) with a reverse transcription oligonucleotide and a reverse transcriptase. In some embodiments, the reverse transcription oligonucleotide is selected from the group consisting of an oligo(dT) primer, a gene-specific primer, and a random oligomer. In some embodiments, the random oligomer is a random hexamer. In some embodiments, the reverse transcription oligonucleotide is bound to a bead. In some embodiments, both the target nucleic acid molecule and non-target nucleic acid molecule (i.e., mRNAs) in a sample (e.g., a single cell) are reverse transcribed, thereby producing the cDNA template (target cDNA products) admixed with non-target cDNA products.

In some embodiments, the method further comprises performing rapid amplification of cDNA ends (RACE) to generate the nucleic acid template (e.g., cDNA template). In some embodiments, 5′-RACE is performed. In some embodiments, 3′-RACE is performed.

### Target Nucleic Acid Molecule

The term “target nucleic acid molecule” refers to a nucleic acid molecule comprising a sequence of contiguous nucleotides that is being analyzed (e.g., for expression level, for sequence information, or for the presence of a mutation).

The target nucleic acid molecule can be, for example, DNA, cDNA or RNA (e.g., noncoding RNA or mRNA). In some embodiments, the target nucleic acid molecule is noncoding RNA. In some embodiments, the target nucleic acid molecule is mRNA. In some embodiments, the target nucleic acid molecule comprises a poly(A) sequence.

The term “sequence” in reference to a nucleic acid, refers to a contiguous series of nucleotides that are joined by covalent bonds (e.g., phosphodiester bonds).

In some embodiments, the target nucleic acid molecule sequence comprises one locus of interest. In some embodiments, the locus of interest comprises a mutation, a polymorphism, an insertion, a deletion, a gene fusion, an edited nucleotide, a modified nucleotide, a transgene or a combination thereof. In some embodiments, the target nucleic acid molecule sequence comprises at least two loci of interest, e.g., 2, 3, 4 or more loci of interest.

### Universal Sequences

In some embodiments, at least a portion of the first universal sequence (e.g., a truncated first universal sequence) acts as a PCR handle for downstream amplification in a sequencer-dependent manner. The first universal sequence may be selected by the cDNA preparation method. In some embodiments, the cDNA preparation method is 10× Genomics and/or the sequencer is an Illumina sequencer. In some embodiments, the truncated first universal sequence is a truncated Illumina Read 1 sequence that is used in 10× Genomics' kits.

In some embodiments, at least a portion of the first universal sequence is the entire first universal sequence. In some embodiments, the entire first universal sequence comprises 5′ ACACTCTTTCCCTACACGACGCTCTTCCGATCT 3′ (SEQ ID NO: 7).

In some embodiments, at least a portion of the first universal sequence is a truncated first universal sequence. In some embodiments, the truncated first universal sequence is missing at least 1 nucleotide at the 5′ end, compared to the entire first universal sequence. In some embodiments, the truncated first universal sequence comprises 5′ CTACACGACGCTCTTCCGATCT 3′ (SEQ ID NO: 5).

The missing first universal sequence refers to a 5′ sequence of the entire first universal sequence missing in the truncated first universal sequence. The missing first universal sequence may comprise 1-20 nucleotides, e.g., 1, 2, 3, 4, 5, 6, 7, 8, 9, 10, 11, 12, 13, 14, 15 or 1-15, 2-15, 2-14, 3-14, 3-13 or 4-13 nucleotides.

The at least a portion of the second universal sequence may be selected based on the downstream sequencing instrument or may be any sequence with a Tₘ high enough for PCR specificity. The at least a portion of the second universal sequence should not match any known naturally occurring sequences. In some embodiments, the at least a portion of the second universal sequence also acts in the circularization event.

In some embodiments, at least a portion of the second universal sequence is a truncated second universal sequence. In some embodiments, at least a portion of the second universal sequence is the entire second universal sequence. In some embodiments, the entire second universal sequence comprises 5′ ACACGTCTGAACTCC 3′ (SEQ ID NO: 8). In some embodiments, the entire second universal sequence comprises 5′ GGAGTTCAGACGTGT 3′ (SEQ ID NO: 9).

### Tag Sequence

In some embodiments, the tag sequence comprises a cell identification tag or a unique molecular identifier (UMI) sequence, or a combination thereof.

As used herein, a “cell identification tag” refers to a sequence of nucleotides that can be incorporated into extension products (e.g., amplicons) and used in sequencing applications to identify the particular cell (e.g., a single cell) or cell type in which the extension product(s) was generated. A cell identification tag can be included in a primer (e.g., an extension primer, such as an oligo(dT) primer, or an amplification primer) for introduction into an extension product (e.g., a RT product, an amplicon). A cell identification tag can be incorporated into an extension product by a suitable nucleic acid polymerase, such as a reverse transcriptase enzyme or a DNA polymerase enzyme.

Non-limiting examples of cell identification tag sequences include

(SEQ ID NO: 10)
5′ AAACCTGAGAAACCAT 3′,
(SEQ ID NO: 11)
5′ AAACCTGAGAAACCGC 3′,
(SEQ ID NO: 12)
5′ AAACCTGAGAAACCTA 3′,
(SEQ ID NO: 13)
5′ AAACCTGAGAAACGAG 3′,
(SEQ ID NO: 14)
5′ AAACCTGAGAAACGCC 3′,
(SEQ ID NO: 15)
5′ AAACCTGAGAAAGTGG 3′,
(SEQ ID NO: 16)
5′ AAACCTGAGAACAACT 3′,
(SEQ ID NO: 17)
5′ AAACCTGAGAACAATC 3′,
(SEQ ID NO: 18)
5′ AAACCTGAGAACTCGG 3′,
and
(SEQ ID NO: 19)
5′ AAACCTGAGAACTGTA 3′.

Additional examples of cell identification tag sequences can be found at https://kb.10×genomics.com/hc/en-us/articles/115004506263-What-is-a-barcode-whitelist-.

“Unique molecular identifiers” or “UMIs”, which are also called “Random Molecular Tags (RMTs),” are sequences of nucleotides that are used to tag a nucleic acid molecule (e.g., prior to amplification) and aid in the identification of duplicates. UMIs are generally random sequences and typically range in size from about 4 to about 20 nucleotides in length. Examples of UMIs are known in the art.

In a well-based system (e.g., 96-well based system), cell identification tag sequences are optional if each well contains 0 or 1 cell.

In some embodiments, the UMI comprises at least one random nucleotide sequence, e.g., 1, 2, 3, 4, 5, 6, 7, 8, 9, 10 or more random nucleotide sequences. The random nucleotide sequence may comprise 1, 2, 3, 4, 5, 6, 7 or more nucleotides. In some embodiments, the random nucleotide is a random hexamer. In some embodiments, the UMI comprises 10 nucleotides. In some embodiments, the UMI comprises 12 nucleotides.

### The First Oligonucleotide

The first oligonucleotide can have a length in the range of about 15 to about 110 nucleotides. For example, the oligonucleotide has a length of about: 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 105 or 110 nucleotides. In some embodiments, the oligonucleotide has a length of about: 20-110, 25-110, 25-100, 30-100, 30-95, 35-95, 35-90, 40-90, 40-85, 50-85, 50-80, 55-80, 55-75, 60-75 or 60-70 nucleotides.

In some embodiments, the first oligonucleotide anneals to a target nucleic acid molecule sequence that is about 0-400 nucleotides 3′ of the locus of interest. For example, about: 0-375, 0-350, 0-325, 0-300, 0-275, 0-250, 0-225, 0-200, 0-175, 0-150, 0-125, 0-100, 0-75, 0-50, or 0-25 nucleotides 3′ of the locus of interest. In some embodiments, the first oligonucleotide anneals to a target nucleic acid molecule sequence that is about 0-150 nucleotides 3′ of the locus of interest.

A person skilled in molecular biology can design the first oligonucleotide appropriate for the sequence platform and kit to be used. In some embodiments, the first oligonucleotide anneals to a target nucleic acid molecule sequence that is about 0-134,000 nucleotides 3′ of the locus of interest (e.g., for Nanopore sequencing). In some embodiments, the first oligonucleotide anneals to a target nucleic acid molecule sequence that is about 0-16,000 nucleotides 3′ of the locus of interest (e.g., for PacBio sequencing). In some embodiments, the first oligonucleotide anneals to a target nucleic acid molecule sequence that is about 0-400 nucleotides 3′ of the locus of interest (e.g., for Illumina sequencing). In some embodiments, the first oligonucleotide anneals to a target nucleic acid molecule sequence that is about 0-150 nucleotides 3′ of the locus of interest (e.g., for Illumina sequencing).

In some embodiments, the first oligonucleotide comprises a target nucleic acid molecule-specific sequence. In some embodiments, the first oligonucleotide comprises, from the 5′ end to the 3′ end, a second universal sequence and a target nucleic acid molecule-specific sequence. In some embodiments, the first oligonucleotide comprises, from the 5′ end to the 3′ end, the missing first universal sequence, a second universal sequence and a target nucleic acid molecule-specific sequence.

In some embodiments, the first oligonucleotide comprises 5′ GGAAAGAGTGT 3′ (SEQ ID NO: 20), 5′ GTGACTGGAGTTCAGACGTGTGCTCTTCCGATCT 3′ (SEQ ID NO: 31) and a target nucleic acid molecule-specific sequence. In some embodiments, the first oligonucleotide comprises 5′ GGAAAGAGTGT 3′ (SEQ ID NO: 20), [i7], 5′ GTGACTGGAGTTCAGACGTGTGCTCTTCCGATCT 3′ (SEQ ID NO: 31) and a target nucleic acid molecule-specific sequence. [i7] is an index (I7) adapter sequence (Illumina, San Diego, CA). In some embodiments, the [i7] sequence is selected from the group consisting of SEQ ID NOs: 34-57.

In some embodiments, the first oligonucleotide comprises 5′ GGAGTTCAGACGTGTGCTCTTCCGATCT 3′ (SEQ ID NO: 21) and a target nucleic acid molecule-specific sequence.

In some embodiments, the target nucleic acid molecule sequence comprises at least two loci of interest. In some embodiments, the first oligonucleotide anneals to a target nucleic acid molecule sequence that is 3′ to the loci of interest (see Alternative A in FIG. 4). In other embodiments, the method comprises the steps of:
a) annealing a plurality of oligonucleotides to the cDNA template;
b) performing cDNA template-directed nucleic acid extensions from the plurality of annealed oligonucleotides to produce a plurality of first extension products comprising all or a portion of target nucleic acid molecule sequences that comprise one or more loci of interest, the tag sequence and at least a portion of the first universal sequence;
c) circularizing the plurality of first extension products to produce a plurality of circularized DNA templates comprising one or more loci of interest, the tag sequence, at least a portion of the first universal sequence and at least a portion of a second universal sequence; and
d) performing circularized DNA template-directed nucleic acid amplifications to produce a plurality of DNA amplicons comprising the one or more loci of interest, the tag sequence, at least a portion of the first universal sequence and at least a portion of the second universal sequence,
   thereby generating a plurality of tagged DNA amplicons for sequencing.

In some embodiments:
a) each of the plurality of first extension products comprises a truncated first universal sequence;
b) each of the plurality of circularized DNA templates comprises the entire first universal sequence and the entire second universal sequence; and
c) each of the plurality of DNA amplicons comprises the entire first universal sequence and the entire second universal sequence.

In some embodiments, performing cDNA template-directed nucleic acid extensions from the plurality of annealed oligonucleotides uses a DNA polymerase lacking 5′→3′exonuclease activity (e.g., Bst DNA Polymerase, Large Fragment, New England Biolabs, Ipswich, MA).

In yet other embodiments, the method comprises the steps of:
a) providing a nucleic acid template comprising a target nucleic acid molecule sequence, a tag sequence and at least a portion of a first universal sequence, wherein the target nucleic acid molecule sequence comprises a locus of interest;
b) performing nucleic acid template-directed nucleic acid amplification to produce a plurality of amplification products by:
   i. contacting the nucleic acid template with a reaction mixture comprising a plurality of oligonucleotide primers and a first primer under conditions in which the plurality of oligonucleotide primers and the first primer anneal to complementary nucleotide sequences in the nucleic acid template, wherein the first primer comprises at least a portion of the first universal sequence; and
   ii. extending each of the plurality of oligonucleotides and the first primer;
c) circularizing the plurality of first amplification products to produce a plurality of circularized DNA templates comprising one or more loci of interest, the tag sequence, at least a portion of the first universal sequence and at least a portion of a second universal sequence; and
d) performing circularized DNA template-directed nucleic acid amplifications to produce a plurality of DNA amplicons comprising the one or more loci of interest, the tag sequence, at least a portion of the first universal sequence and at least a portion of the second universal sequence,
   thereby generating a plurality of tagged DNA amplicons for sequencing.

In some embodiments:
a) each of the plurality of circularized DNA templates comprises the entire first universal sequence and the entire second universal sequence; and
b) each of the plurality of DNA amplicons comprises the entire first universal sequence and the entire second universal sequence.

In some embodiments, performing nucleic acid template-directed nucleic acid amplification uses a DNA polymerase lacking 5′→3′exonuclease activity (e.g., Bst DNA Polymerase, Large Fragment, New England Biolabs, Ipswich, MA).

### Nucleic Acid Template-Directed Nucleic Acid Extension

In some embodiments, the first extension product comprises, from the 5′ end to the 3′ end, all or a portion of the target nucleic acid molecule sequence comprising the locus of interest, the tag sequence and at least a portion of the first universal sequence. In some embodiments, the first extension product comprises, from the 5′ end to the 3′ end, all or a portion of the target nucleic acid molecule sequence comprising the locus of interest, the poly(A) sequence, the tag sequence and at least a portion of the first universal sequence.

In some embodiments, the first extension product comprises, from the 5′ end to the 3′ end, at least a portion of the second universal sequence, all or a portion of the target nucleic acid molecule sequence comprising the locus of interest, the tag sequence and at least a portion of the first universal sequence. In some embodiments, the first extension product comprises, from the 5′ end to the 3′ end, at least a portion of the second universal sequence, all or a portion of the target nucleic acid molecule sequence comprising the locus of interest, the poly(A) sequence, the tag sequence and at least a portion of the first universal sequence.

In some embodiments, the first extension product comprises, from the 5′ end to the 3′ end, all or a portion of the target nucleic acid molecule sequence comprising the locus of interest and the TSO2 sequence. In some embodiments, the first extension product comprises, from the 5′ end to the 3′ end, at least a portion of the second universal sequence, all or a portion of the target nucleic acid molecule sequence comprising the locus of interest and the TSO2 sequence.

In some embodiments, the at least a portion of the first universal sequence is a truncated first universal sequence. In some embodiments, the at least a portion of the second universal sequence is the entire second universal sequence. In some embodiments, the at least a portion of the first universal sequence is a truncated first universal sequence and the at least a portion of the second universal sequence is the entire second universal sequence.

In some embodiments, the first extension product comprises the missing first universal sequence at the 5′ end. In some embodiments, the first extension product comprises, from the 5′ end to the 3′ end, the missing first universal sequence, the second universal sequence, all or a portion of the target nucleic acid molecule sequence comprising the locus of interest, the tag sequence and the truncated first universal sequence. In some embodiments, the first extension product comprises, from the 5′ end to the 3′ end, the missing first universal sequence, the second universal sequence, all or a portion of the target nucleic acid molecule sequence comprising the locus of interest, the poly(A) sequence, the tag sequence and the truncated first universal sequence. In some embodiments, the first extension product comprises, from the 5′ end to the 3′ end, the missing first universal sequence, a second universal sequence, all or a portion of the target nucleic acid molecule sequence comprising the locus of interest and the TSO2 sequence.

In some embodiments, the first extension product comprises all of the target nucleic acid molecule sequence. In some embodiments, the first extension product comprises a portion of the target nucleic acid molecule sequence comprising the locus of interest.

### First Extension Product-Directed Nucleic Acid Amplification

In some embodiments, the method further comprises performing first extension product-directed nucleic acid amplification to amplify the first extension product. In some embodiments, the first extension product-directed nucleic acid amplification is performed using the first oligonucleotide and a first primer.

In some embodiments, the first primer comprises at least a portion of the entire first universal sequence. In some embodiments, the first primer comprises at least a portion of the truncated first universal sequence.

In some embodiments, the first primer further comprises at least a portion of a second universal sequence. In some embodiments, the first primer comprises, from the 5′ end to the 3′ end, at least a portion of a second universal sequence, the missing first universal sequence and at least a portion of the truncated first universal sequence. In some embodiments, the first primer comprises, from the 5′ end to the 3′ end, at least a portion of a second universal sequence and at least a portion of the first universal sequence. In some embodiments, the at least a portion of the second universal sequence is the entire second universal sequence. In some embodiments, the at least a portion of the second universal sequence is a truncated second universal sequence. In some embodiments, the first primer comprises, from the 5′ end to the 3′ end, the entire second universal sequence, the missing first universal sequence and at least a portion of the truncated first universal sequence.

In some embodiments, the first primer further comprises [i7]. In some embodiments, the first primer comprises, from the 5′ end to the 3′ end, at least a portion of a second universal sequence, [i7], the missing first universal sequence and at least a portion of the truncated first universal sequence. In some embodiments, the first primer comprises, from the 5′ end to the 3′ end, at least a portion of a second universal sequence, [i7] and at least a portion of the first universal sequence. In some embodiments, the first primer comprises, from the 5′ end to the 3′ end, the entire second universal sequence, [i7], the missing first universal sequence and at least a portion of the truncated first universal sequence. In some embodiments, the first primer comprises, from 5′ end to the 3′ end, [i7] and at least a portion of the entire first universal sequence. In some embodiments, the first primer comprises, from 5′ end to the 3′ end, [i7] and at least a portion of the truncated first universal sequence. In some embodiments, the [i7] sequence is selected from the group consisting of SEQ ID NOs: 34-57.

In some embodiments, the first primer is a first reverse primer. In some embodiments, the first reverse primer comprises at least a portion of the truncated first universal sequence. In some embodiments, the first reverse primer comprises, from the 5′ end to the 3′ end, the missing first universal sequence and at least a portion of the truncated first universal sequence. In some embodiments, the first reverse primer comprises, from the 5′ end to the 3′ end, a second universal sequence, the missing first universal sequence and at least a portion of the truncated first universal sequence. In some embodiments, the first reverse primer comprises, from the 5′ end to the 3′ end, a second universal sequence, [i7], the missing first universal sequence and at least a portion of the truncated first universal sequence. In some embodiments, the first reverse primer comprises, from the 5′ end to the 3′ end, 5′ ACACGTCTGAACTCCAGTCAC 3′ (SEQ ID NO: 22) and 5′ ACACTCTTTCCCTACACGACGCTCTTCCGATCT 3′ (SEQ ID NO: 7). In some embodiments, the first reverse primer comprises, from the 5′ end to the 3′ end, 5′ ACACGTCTGAACTCCAGTCAC 3′ (SEQ ID NO: 22), [i7], and 5′ ACACTCTTTCCCTACACGACGCTCTTCCGATCT 3′ (SEQ ID NO: 7).

In some embodiments, the first primer is a first forward primer. In some embodiments, the first forward primer comprises at least a portion of the truncated first universal sequence. In some embodiments, the first forward primer comprises, from the 5′ end to the 3′ end, the missing first universal sequence and at least a portion of the truncated first universal sequence. In some embodiments, the first forward primer comprises, from the 5′ end to the 3′ end, a second universal sequence, the missing first universal sequence and at least a portion of the truncated first universal sequence. In some embodiments, the first forward primer comprises, from the 5′ end to the 3′ end, a second universal sequence, [i7], the missing first universal sequence and at least a portion of the truncated first universal sequence. In some embodiments, the first forward primer comprises, from the 5′ end to the 3′ end, 5′ ACACGTCTGAACTCCAGTCAC 3′ (SEQ ID NO: 22), [i7], and 5′ ACACTCTTTCCCTACACGACGCTCTTCCGATCT 3′ (SEQ ID NO: 7).

In some embodiments (3′ library), the first extension product-directed nucleic acid amplification product comprises, from the 5′ end to the 3′ end, 5′ GGAGTTCAGACGTGTGCTCTTCCGATCT 3′ (SEQ ID NO: 21), all or a portion of the target nucleic acid molecule sequence comprising the locus of interest, poly(A), the cell identification tag sequence, UMI, 5′ AGATCGGAAGAGCGTCGTGTAGGGAAAGAGTGT 3′ (SEQ ID NO: 23), [i7] and 5′ GTGACTGGAGTTCAGACGTGT 3′ (SEQ ID NO: 24).

In some embodiments (5′ library), the first extension product-directed nucleic acid amplification product comprises, from the 5′ end to the 3′ end, 5′ ACACGTCTGAACTCCAGTCAC 3′ (SEQ ID NO: 22), [i7], 5′ ACACTCTTTCCCTACACGACGCTCTTCCGATCT 3′ (SEQ ID NO: 7), cell identification tag sequence, 5′ TTTCTTATATGGG 3′ (SEQ ID NO: 25), all or a portion of the target nucleic acid molecule sequence comprising the locus of interest and 5′ AGATCGGAAGAGCACACGTCTGAACTCC 3′ (SEQ ID NO: 26).

In some embodiments, the first oligonucleotide or the first primer further comprises, at the 3′ end, an RNA base followed by a blocking domain; and amplifying the first extension product comprises performing an RNase H-dependent PCR. In some embodiments, performing an RNase H-dependent PCR increases the specificity of the first extension product-directed nucleic acid amplification. Details of this method are described, for example, in International Application No. PCT/IB2019/001398, the contents of which are incorporated herein by reference.

### Nucleic Acid Template-Directed Nucleic Acid Amplification

In some embodiments, the first oligonucleotide comprises a target nucleic acid molecule-specific sequence. In some embodiments, the first oligonucleotide comprises, from the 5′ end to the 3′ end, at least a portion of a second universal sequence and a target nucleic acid molecule-specific sequence. In some embodiments, the first oligonucleotide comprises, from the 5′ end to the 3′ end, the missing first universal sequence, at least a portion of a second universal sequence and a target nucleic acid molecule-specific sequence. In some embodiments, at least a portion of a second universal sequence is the entire second universal sequence. In some embodiments, at least a portion of a second universal sequence is a truncated second universal sequence.

In some embodiments, the first primer comprises at least a portion of the entire first universal sequence. In some embodiments, the first primer comprises at least a portion of the truncated first universal sequence. In some embodiments, the first primer comprises, from the 5′ end to the 3′ end, the missing first universal sequence and at least a portion of the truncated first universal sequence.

In some embodiments, the first primer further comprises at least a portion of the second universal sequence. In some embodiments, the first primer comprises, from the 5′ end to the 3′ end, at least a portion of a second universal sequence, the missing first universal sequence and at least a portion of the truncated first universal sequence. In some embodiments, the first primer comprises, from the 5′ end to the 3′ end, at least a portion of a second universal sequence and at least a portion of the entire first universal sequence. In some embodiments, at least a portion of a second universal sequence is the entire second universal sequence. In some embodiments, at least a portion of a second universal sequence is a truncated second universal sequence. In some embodiments, the first primer comprises, from the 5′ end to the 3′ end, the entire second universal sequence, the missing first universal sequence and at least a portion of the truncated first universal sequence.

In some embodiments, the first primer further comprises [i7]. In some embodiments, the first primer comprises, from the 5′ end to the 3′ end, [i7], the missing first universal sequence and at least a portion of the truncated first universal sequence. In some embodiments, the first primer comprises, from the 5′ end to the 3′ end, [i7] and at least a portion of the entire first universal sequence. In some embodiments, the first primer comprises, from the 5′ end to the 3′ end, at least a portion of a second universal sequence, [i7], the missing first universal sequence and at least a portion of the truncated first universal sequence. In some embodiments, the first primer comprises, from the 5′ end to the 3′ end, at least a portion of a second universal sequence, [i7] and at least a portion of the entire first universal sequence. In some embodiments, at least a portion of a second universal sequence is a truncated second universal sequence. In some embodiments, the first primer comprises, from the 5′ end to the 3′ end, the entire second universal sequence, [i7], the missing first universal sequence and at least a portion of the truncated first universal sequence.

In some embodiments:
a) the first oligonucleotide comprises, from the 5′ end to the 3′ end, the entire second universal sequence and a target nucleic acid molecule-specific sequence; and
b) the first primer comprises, from the 5′ end to the 3′ end, the entire second universal sequence, [i7], the missing first universal sequence and at least a portion of the truncated first universal sequence.

In some embodiments, the first primer is a first reverse primer. In some embodiments, the first reverse primer comprises, from the 5′ end to the 3′ end, 5′ ACACGTCTGAACTCCAGTCAC 3′ (SEQ ID NO: 22), [i7], and 5′ ACACTCTTTCCCTACACGACGCTCTTCCGATCT 3′ (SEQ ID NO: 7).

In some embodiments, the first primer is a first forward primer. In some embodiments, the first forward primer comprises, from the 5′ end to the 3′ end, 5′ ACACGTCTGAACTCCAGTCAC 3′ (SEQ ID NO: 22), [i7], and 5′ ACACTCTTTCCCTACACGACGCTCTTCCGATCT 3′ (SEQ ID NO: 7).

In some embodiments, the first amplification product comprises, from the 5′ end to the 3′ end, at least a portion of the second universal sequence, all or a portion of the target nucleic acid molecule sequence comprising the locus of interest, the tag sequence, at least a portion of the first universal sequence, [i7] and at least a portion of the second universal sequence. In some embodiments, the first amplification product comprises, from the 5′ end to the 3′ end, at least a portion of the second universal sequence, all or a portion of the target nucleic acid molecule sequence comprising the locus of interest, the poly(A) sequence, the tag sequence, at least a portion of the first universal sequence, [i7] and at least a portion of the second universal sequence (see, e.g., FIG. 1B). In some embodiments, the first amplification product comprises, from the 5′ end to the 3′ end, at least a portion of the second universal sequence, all or a portion of the target nucleic acid molecule sequence comprising the locus of interest, the TSO2 sequence, [i7] and at least a portion of the second universal sequence (see, e.g., FIG. 2).

In some embodiments, the first amplification product comprises, from the 5′ end to the 3′ end, all or a portion of the target nucleic acid molecule sequence comprising the locus of interest, the tag sequence, at least a portion of the first universal sequence, [i7] and at least a portion of the second universal sequence. In some embodiments, the first amplification product comprises, from the 5′ end to the 3′ end, all or a portion of the target nucleic acid molecule sequence comprising the locus of interest, the poly(A) sequence, the tag sequence, at least a portion of the first universal sequence, [i7] and at least a portion of the second universal sequence. In some embodiments, the first amplification product comprises, from the 5′ end to the 3′ end, all or a portion of the target nucleic acid molecule sequence comprising the locus of interest, the TSO2 sequence, [i7] and at least a portion of the second universal sequence.

In some embodiments, the first amplification product comprises, from the 5′ end to the 3′ end, at least a portion of the second universal sequence, all or a portion of the target nucleic acid molecule sequence comprising the locus of interest, the tag sequence, at least a portion of the first universal sequence and [i7]. In some embodiments, the first amplification product comprises, from the 5′ end to the 3′ end, at least a portion of the second universal sequence, all or a portion of the target nucleic acid molecule sequence comprising the locus of interest, the poly(A) sequence, the tag sequence, at least a portion of the first universal sequence and [i7]. In some embodiments, the first amplification product comprises, from the 5′ end to the 3′ end, at least a portion of the second universal sequence, all or a portion of the target nucleic acid molecule sequence comprising the locus of interest, the TSO2 sequence and [i7].

In some embodiments, the first amplification product comprises, from the 5′ end to the 3′ end, at least a portion of the second universal sequence, all or a portion of the target nucleic acid molecule sequence comprising the locus of interest, the tag sequence, at least a portion of the first universal sequence and at least a portion of the second universal sequence. In some embodiments, the first amplification product comprises, from the 5′ end to the 3′ end, at least a portion of the second universal sequence, all or a portion of the target nucleic acid molecule sequence comprising the locus of interest, the poly(A) sequence, the tag sequence, at least a portion of the first universal sequence and at least a portion of the second universal sequence. In some embodiments, the first amplification product comprises, from the 5′ end to the 3′ end, the second universal sequence, all or a portion of the target nucleic acid molecule sequence comprising the locus of interest, the TSO2 sequence and at least a portion of the second universal sequence.

In some embodiments, the first amplification product comprises, from the 5′ end to the 3′ end, all or a portion of the target nucleic acid molecule sequence comprising the locus of interest, the tag sequence, at least a portion of the first universal sequence and at least a portion of the second universal sequence. In some embodiments, the first amplification product comprises, from the 5′ end to the 3′ end, all or a portion of the target nucleic acid molecule sequence comprising the locus of interest, the poly(A) sequence, the tag sequence, at least a portion of the first universal sequence and at least a portion of the second universal sequence. In some embodiments, the first amplification product comprises, from the 5′ end to the 3′ end, all or a portion of the target nucleic acid molecule sequence comprising the locus of interest, the TSO2 sequence and at least a portion of the second universal sequence.

In some embodiments, the first amplification product comprises, from the 5′ end to the 3′ end, at least a portion of the second universal sequence, all or a portion of the target nucleic acid molecule sequence comprising the locus of interest, the tag sequence and at least a portion of the first universal sequence. In some embodiments, the first amplification product comprises, from the 5′ end to the 3′ end, at least a portion of the second universal sequence, all or a portion of the target nucleic acid molecule sequence comprising the locus of interest, the poly(A) sequence, the tag sequence and at least a portion of the first universal sequence. In some embodiments, the first amplification product comprises, from the 5′ end to the 3′ end, at least a portion of the second universal sequence, all or a portion of the target nucleic acid molecule sequence comprising the locus of interest and the TSO2 sequence.

In some embodiments, the at least a portion of the first universal sequence is the entire first universal sequence. In some embodiments, the at least a portion of the first universal sequence is a truncated first universal sequence. In some embodiments, the at least a portion of the second universal sequence is the entire second universal sequence. In some embodiments, the at least a portion of the second universal sequence is a truncated second universal sequence. In some embodiments, the at least a portion of the first universal sequence is the entire first universal sequence and the at least a portion of the second universal sequence is the entire second universal sequence. In some embodiments, the at least a portion of the first universal sequence is the entire first universal sequence and the at least a portion of the second universal sequence is the truncated second universal sequence. In some embodiments, the at least a portion of the first universal sequence is a truncated first universal sequence and the at least a portion of the second universal sequence is the entire second universal sequence. In some embodiments, the at least a portion of the first universal sequence is a truncated first universal sequence and the at least a portion of the second universal sequence is a truncated second universal sequence.

In some embodiments, the first amplification product comprises all of the target nucleic acid molecule sequence. In some embodiments, the first amplification product comprises a portion of the target nucleic acid molecule sequence comprising the locus of interest.

In some embodiments (3′ library), the first amplification product comprises, from the 5′ end to the 3′ end, 5′ GGAGTTCAGACGTGTGCTCTTCCGATCT 3′ (SEQ ID NO: 21), all or a portion of the target nucleic acid molecule sequence comprising the locus of interest, poly(A), the cell identification tag sequence, UMI, 5′ AGATCGGAAGAGCGTCGTGTAGGGAAAGAGTGT 3′ (SEQ ID NO: 23), [i7] and 5′ GTGACTGGAGTTCAGACGTGT 3′ (SEQ ID NO: 24).

In some embodiments (5′ RNA-Seq library), the first amplification product comprises, from the 5′ end to the 3′ end, 5′ ACACGTCTGAACTCCAGTCAC 3′ (SEQ ID NO: 22), [i7], 5′ ACACTCTTTCCCTACACGACGCTCTTCCGATCT 3′ (SEQ ID NO: 7), cell identification tag sequence, 5′ TTTCTTATATGGG 3′ (SEQ ID NO: 25), all or a portion of the target nucleic acid molecule sequence comprising the locus of interest and 5′ AGATCGGAAGAGCACACGTCTGAACTCC 3′ (SEQ ID NO: 26).

In some embodiments, the first oligonucleotide or the first primer further comprises, at the 3′ end, an RNA base followed by a blocking domain; and amplifying the first extension product comprises performing an RNase H-dependent PCR. In some embodiments, performing an RNase H-dependent PCR increases specificity of the nucleic acid template-directed nucleic acid amplification. Details of this method are described, for example, in International Application No. PCT/IB2019/001398, the contents of which are incorporated herein by reference.

Circularizing the First Extension and/or Amplification Product

In some embodiments, the method comprises a single circularizing step. In some embodiments, the method, which requires a single circularizing step, increases efficiency, reduces data dropout, or a combination thereof.

In some embodiments, circularizing the first extension and/or amplification product comprises an intramolecular ligation mediated by Gibson assembly, splint ligation, or a ligation using a thermostable ATP-dependent ligase. In some embodiments, circularizing the first extension and/or amplification product comprises an intramolecular ligation mediated by Gibson assembly. In some embodiments, circularizing the first extension and/or amplification product comprises an intramolecular ligation mediated by splint ligation. In some embodiments, circularizing the first extension and/or amplification product comprises an intramolecular ligation mediated by a ligation using a thermostable ATP-dependent ligase. Non-limiting examples of thermostable ATP-dependent ligases include CIRCL-LIGASE and T4 DNA ligase.

The circularized DNA template can be single stranded or double stranded.

In some embodiments, the circularized DNA template comprises:
a) at least a portion of the second universal sequence;
b) all or a portion of the target nucleic acid molecule sequence comprising the locus of interest;
c) the tag sequence; and
d) at least a portion of the first universal sequence.

In some embodiments, the circularized DNA template comprises:
a) at least a portion of the second universal sequence;
b) all or a portion of the target nucleic acid molecule sequence comprising the locus of interest;
c) the tag sequence;
d) at least a portion of the first universal sequence; and
e) [i7].

In some embodiments, the circularized DNA template comprises:
a) at least a portion of the second universal sequence;
b) all or a portion of the target nucleic acid molecule sequence comprising the locus of interest;
c) the poly(A) sequence;
d) the tag sequence; at least a portion of the first universal sequence; and
e) at least a portion of the second universal sequence.

In some embodiments, the circularized DNA template comprises:
a) at least a portion of the second universal sequence;
b) all or a portion of the target nucleic acid molecule sequence comprising the locus of interest;
c) the poly(A) sequence;
d) the tag sequence;
e) at least a portion of the first universal sequence; and
f) [i7].

In some embodiments, the circularized DNA template comprises:
a) at least a portion of the second universal sequence;
b) all or a portion of the target nucleic acid molecule sequence comprising the locus of interest;
c) and the TSO2 sequence.

In some embodiments, the circularized DNA template comprises:
a) at least a portion of the second universal sequence;
b) all or a portion of the target nucleic acid molecule sequence comprising the locus of interest;
c) the TSO2 sequence; and
d) [i7].

In some embodiments, the at least a portion of the first universal sequence is the entire first universal sequence. In some embodiments, the at least a portion of the second universal sequence is the entire second universal sequence. In some embodiments, the at least a portion of the first universal sequence is the entire first universal sequence and the at least a portion of the second universal sequence is the entire second universal sequence. In some embodiments, the at least a portion of the first universal sequence is a truncated first universal sequence. In some embodiments, the at least a portion of the second universal sequence is a truncated second universal sequence.

### Circularized DNA Template-Directed Nucleic Acid Amplification

In some embodiments, circularized DNA template-directed nucleic acid amplification comprises amplifying a portion of the circularized DNA template with a high-fidelity DNA Polymerase. Non-limiting examples of high-fidelity DNA Polymerases include Q5® High-Fidelity DNA Polymerase (New England Biolabs Inc., Ipswich, MA) and KOD DNA Polymerase (Sigma Aldrich, St. Louis, MO).

In some embodiments, performing circularized DNA template-directed nucleic acid amplification uses a second forward primer and a second reverse primer.

In some embodiments, the second forward primer or the second reverse primer comprises a target nucleic acid molecule-specific sequence, all or a portion of the tag sequence, all or a portion of the first universal sequence, a poly(A) sequence, or a combination thereof.

In some embodiments, the second forward primer comprises a target nucleic acid molecule-specific sequence. In some embodiments, the second forward primer comprises, from the 5′ end to the 3′ end, the Illumina P7 sequence and the target nucleic acid molecule-specific sequence. In some embodiments, the second forward primer comprises a poly(A) sequence. In some embodiments, the second forward primer comprises, from the 5′ end to the 3′ end, the Illumina P7 sequence and the poly(A) sequence. In some embodiments, the second forward primer comprises the TSO2 sequence. In some embodiments, the second forward primer comprises, from the 5′ end to the 3′ end, the Illumina P7 sequence and the TSO2 sequence.

In some embodiments, the second reverse primer comprises a target nucleic acid molecule-specific sequence. In some embodiments, the second reverse primer comprises, from the 5′ end to the 3′ end, the Illumina P5 sequence and the target nucleic acid molecule-specific sequence.

In some embodiments:
a) the second forward primer further comprises a sequence that is reversed and complementary to a first immobilizing oligonucleotide sequence; and
b) the second reverse primer further comprises a sequence that is reversed and complementary to a second immobilizing oligonucleotide sequence.

In some embodiments, the first and the second immobilizing oligos are immobilized on the Illumina flow cell.

In some embodiments, the first and the second immobilizing oligonucleotide sequences comprise:

a)
(SEQ ID NO: 27)
CCTCTCTATGGGCAGTCGGTGAT
and
(SEQ ID NO: 28)
CCATCTCATCCCTGCGTGTCTCCGACTCAG, respectively;
b)
(SEQ ID NO: 28)
CCATCTCATCCCTGCGTGTCTCCGACTCAG
and
(SEQ ID NO: 27)
CCTCTCTATGGGCAGTCGGTGAT, respectively;
or
c)
(SEQ ID NO: 29)
CAAGCAGAAGACGGCATACGAGAT
and
(SEQ ID NO: 30)
AATGATACGGCGACCACCGAGATCTACAC, respectively.

In some embodiments (3′ RNA-Seq library):
a) the second forward primer comprises, from the 5′ end to the 3′ end, 5′ AATGATACGGCGACCACCGAGATCTACAC 3′ (SEQ ID NO: 30) and a target nucleic acid molecule-specific sequence; and
b) the second reverse primer comprises, from the 5′ end to the 3′ end, 5′ CAAGCAGAAGACGGCATACGAGAT 3′ (SEQ ID NO: 29) and a poly(A) sequence or a target nucleic acid molecule-specific sequence.

In some embodiments (5′ RNA-Seq library):
a) the second forward primer comprises, from the 5′ end to the 3′ end, 5′ AATGATACGGCGACCACCGAGATCTACAC 3′ (SEQ ID NO: 30) and a target nucleic acid molecule-specific sequence; and
b) the second reverse primer comprises, from the 5′ end to the 3′ end, 5′ CAAGCAGAAGACGGCATACGAGAT 3′ (SEQ ID NO: 29) and the TSO2 sequence or a target nucleic acid molecule-specific sequence.

In some embodiments, the second forward primer or the second reverse primer further comprises, at the 3′ end, an RNA base followed by a blocking domain; and amplifying the circularized DNA template comprises performing an RNase H-dependent PCR (rhPCR). In some embodiments, the rhPCR is performed to increase PCR specificity. Details of this method are described, for example, in International Application No. PCT/IB2019/001398, the contents of which are incorporated herein by reference.

### The DNA Amplicon

In some embodiments, the DNA amplicon comprises, from the 5′ end to the 3′ end, the Illumina P5 sequence, the locus of interest, at least a portion of the second universal sequence, [i7], at least a portion of the first universal sequence, the tag sequence, the poly(T) sequence and the Illumina P7 sequence (see, e.g., FIG. 1A). In some embodiments, the DNA amplicon comprises, from the 5′ end to the 3′ end, the Illumina P5 sequence, the locus of interest, at least a portion of the second universal sequence, at least a portion of the first universal sequence, the tag sequence, the poly(T) sequence and the Illumina P7 sequence.

In some embodiments, the DNA amplicon comprises, from the 5′ end to the 3′ end, the Illumina P5 sequence, the locus of interest, at least a portion of the second universal sequence, [i7], the TSO2 sequence and the Illumina P7 sequence (see, e.g., FIG. 2). In some embodiments, the DNA amplicon comprises, from the 5′ end to the 3′ end, the Illumina P5 sequence, the locus of interest, at least a portion of the second universal sequence the TSO2 sequence and the Illumina P7 sequence.

In some embodiments, the DNA amplicon comprises, from the 5′ end to the 3′ end, the Illumina P5 sequence, the locus of interest, at least a portion of the second universal sequence, [i7], at least a portion of the first universal sequence, the tag sequence and the Illumina P7 sequence. In some embodiments, the DNA amplicon comprises, from the 5′ end to the 3′ end, the Illumina P5 sequence, the locus of interest, at least a portion of the second universal sequence, at least a portion of the first universal sequence, the tag sequence and the Illumina P7 sequence.

In some embodiments, the at least a portion of the first universal sequence is the entire first universal sequence. In some embodiments, the at least a portion of the second universal sequence is the entire second universal sequence. In some embodiments, the at least a portion of the first universal sequence is the entire first universal sequence and the at least a portion of the second universal sequence is the entire second universal sequence. In some embodiments, the at least a portion of the first universal sequence is a truncated first universal sequence. In some embodiments, the at least a portion of the second universal sequence is a truncated second universal sequence.

In some embodiments, the DNA amplicon is between about 100 base pairs and about 5,000 base pairs in length. For example, about: 100-4,500, 150-4,500, 150-4,000, 200-4,000, 200-3,500, 250-3,500, 250-3,000, 300-3,000, 300-2,500, 350-2,500, 350-2,000, 350-1,500, 350-1,000, 300-1,000, 300-800, 400-800 or 400-600 base pairs in length. In some embodiments, the DNA amplicon is between about 200 base pairs and about 500 base pairs in length.

In some embodiments, the DNA amplicon is less than about 5,000 base pairs in length. For example, less than about: 4,500, 4,000, 3,500, 3,000, 2,500, 2,000, 1,500, 1,000, 900, 800, 700, 600, 500, 400, 300 or 200 base pairs in length.

In some embodiments (3′ RNA-Seq library), the DNA amplicon comprises, from the 5′ end to the 3′ end, 5′ AATGATACGGCGACCACCGAGATCTACAC 3′ (SEQ ID NO: 30), the locus of interest, 5′ AGATCGGAAGAGCACACGTCTGAACTCCAGTCAC 3′ (SEQ ID NO: 32), [i7], 5′ ACACTCTTTCCCTACACGACGCTCTTCCGATCT 3′ (SEQ ID NO: 7), cell identification tag sequence, UMI, oligo(dT), reverse and complementary sequence of target nucleic acid molecule-specific sequence and 5′ ATCTCGTATGCCGTCTTCTGCTTG 3′ (SEQ ID NO: 33).

In some embodiments (5′ RNA-Seq library), the DNA amplicon comprises, from the 5′ end to the 3′ end, 5′ AATGATACGGCGACCACCGAGATCTACAC 3′ (SEQ ID NO: 30), the locus of interest, 5′ AGATCGGAAGAGCACACGTCTGAACTCCAGTCAC 3′ (SEQ ID NO: 32), [i7], 5′ ACACTCTTTCCCTACACGACGCTCTTCCGATCT 3′ (SEQ ID NO: 7), cell identification tag sequence, UMI, TSO2, target nucleic acid molecule-specific sequence and 5′ ATCTCGTATGCCGTCTTCTGCTTG 3′ (SEQ ID NO: 33).

### Sequencing

In some embodiments, the method is used to make a sequencing library.

In some embodiments, the method further comprises sequencing the DNA amplicon.

In some embodiments, the sequencing is performed using a first universal sequence-specific primer, a second universal sequence-specific primer, an I7 sequence-specific primer or a combination of the foregoing. In some embodiments, the first universal sequence-specific primer comprises all or a portion of ACACTCTTTCCCTACACGACGCTCTTCCGATCT (SEQ ID NO: 7). In some embodiments, the second universal sequence-specific primer comprises all or a portion of GTGACTGGAGTTCAGACGTGTGCTCTTCCGATCT (SEQ ID NO: 31). In some embodiments, the I7 sequence-specific primer comprises all or a portion of AGATCGGAAGAGCACACGTCTGAACTCCAGTCAC (SEQ ID NO: 32).

In some embodiments, the method identifies a modified nucleotide. Identifying a modified nucleotide may require pre-treatment of the sample.

In another aspect, the invention provides a tagged DNA amplicon for sequencing, comprising, from the 5′ end to the 3′ end, a locus of interest, at least a portion of a second universal sequence, at least a portion of a first universal sequence and a tag sequence. In another aspect, the invention provides a tagged DNA amplicon for sequencing, comprising, from the 5′ end to the 3′ end, a locus of interest, a second universal sequence, at least a portion of a first universal sequence and a tag sequence. In another aspect, the invention provides a tagged DNA amplicon for sequencing, comprising, from the 5′ end to the 3′ end, a locus of interest, at least a portion of a second universal sequence, a first universal sequence and a tag sequence. In some embodiments, the tagged DNA amplicon comprises, from the 5′ end to the 3′ end, the locus of interest, the second universal sequence, the first universal sequence and the tag sequence.

In some embodiments, the tagged DNA amplicon comprises, from the 5′ end to the 3′ end, an Illumina P5 sequence, the locus of interest, at least a portion of the second universal sequence, at least a portion of the first universal sequence, the tag sequence and the Illumina P7 sequence. In some embodiments, the tagged DNA amplicon comprises, from the 5′ end to the 3′ end, an Illumina P5 sequence, the locus of interest, the second universal sequence, at least a portion of the first universal sequence, the tag sequence and the Illumina P7 sequence. In some embodiments, the tagged DNA amplicon comprises, from the 5′ end to the 3′ end, an Illumina P5 sequence, the locus of interest, at least a portion of the second universal sequence, the first universal sequence, the tag sequence and the Illumina P7 sequence. In some embodiments, the tagged DNA amplicon comprises, from the 5′ end to the 3′ end, an Illumina P5 sequence, the locus of interest, the second universal sequence, the first universal sequence, the tag sequence and the Illumina P7 sequence.

In some embodiments, the tagged DNA amplicon comprises, from the 5′ end to the 3′ end, an Illumina P5 sequence, the locus of interest, at least a portion of the second universal sequence, [i7], at least a portion of the first universal sequence, the tag sequence and the Illumina P7 sequence. In some embodiments, the tagged DNA amplicon comprises, from the 5′ end to the 3′ end, an Illumina P5 sequence, the locus of interest, the second universal sequence, [i7], at least a portion of the first universal sequence, the tag sequence and the Illumina P7 sequence. In some embodiments, the tagged DNA amplicon comprises, from the 5′ end to the 3′ end, an Illumina P5 sequence, the locus of interest, at least a portion of the second universal sequence, [i7], the first universal sequence, the tag sequence and the Illumina P7 sequence. In some embodiments, the tagged DNA amplicon comprises, from the 5′ end to the 3′ end, an Illumina P5 sequence, the locus of interest, the second universal sequence, [i7], the first universal sequence, the tag sequence and the Illumina P7 sequence.

In some embodiments, the tagged DNA amplicon comprises, from the 5′ end to the 3′ end, an Illumina P5 sequence, the locus of interest, at least a portion of the second universal sequence, [i7], at least a portion of the first universal sequence, the tag sequence, a poly(T) sequence and the Illumina P7 sequence. In some embodiments, the tagged DNA amplicon comprises, from the 5′ end to the 3′ end, an Illumina P5 sequence, the locus of interest, the second universal sequence, [i7], at least a portion of the first universal sequence, the tag sequence, a poly(T) sequence and the Illumina P7 sequence. In some embodiments, the tagged DNA amplicon comprises, from the 5′ end to the 3′ end, an Illumina P5 sequence, the locus of interest, at least a portion of the second universal sequence, [i7], the first universal sequence, the tag sequence, a poly(T) sequence and the Illumina P7 sequence. In some embodiments, the tagged DNA amplicon comprises, from the 5′ end to the 3′ end, an Illumina P5 sequence, the locus of interest, the second universal sequence, [i7], the first universal sequence, the tag sequence, a poly(T) sequence and the Illumina P7 sequence.

In some embodiments, the tagged DNA amplicon comprises, from the 5′ end to the 3′ end, an Illumina P5 sequence, the locus of interest, at least a portion of the second universal sequence, [i7], a second template switching oligo (TSO2) sequence and the Illumina P7 sequence. In some embodiments, the tagged DNA amplicon comprises, from the 5′ end to the 3′ end, an Illumina P5 sequence, the locus of interest, the second universal sequence, [i7], a second template switching oligo (TSO2) sequence and the Illumina P7 sequence.

### EXAMPLES

### Example 1

### Methods

As illustrated in FIG. 1A, a cDNA from HEK 293T cell line was reverse transcribed using an oligo dT primer containing partial Illumina Read 1. First gene-specific oligonucleotide tagged with partial Read 1, i7 and Read 2 is used to generate a single-stranded amplicon. The molecular structure of the single-stranded amplicon is depicted in “3.” Splint ligation is performed using a complementary RNA oligonucleotide to bridge the 5′ and 3′ ends. Splint ligation generates a single-stranded circularized template as shown in “4.” PCR with P5-GSP and P7-GSP or P7-polyA yields the final sequencing library. The generated cDNA has a simplified molecular structure similar to cDNA prepared with the 10× Genomics Chromium 3′ gene expression kit (10× Genomics, Pleasanton, CA).

Following cDNA synthesis, hemi-nested gene-specific PCR was performed using KAPA HiFi DNA polymerase (Kapa Biosystems Inc., Wilmington, MA). The primer pair consists of a gene-specific primer that primes 1-300 base pairs (e.g., approximately 50 base pairs) upstream of the locus-of-interest and a Read 1-specific primer priming onto the 3′-end of the cDNA. Both primers comprise the Illumina Read 2 sequence at the 5′ ends, which provides the homology arms necessary for subsequent Gibson assembly. The hemi-nested PCR amplicon was cleaned up with Zymo DNA clean and concentrate (DCC-5) columns (Zymo Research, Irvine, CA).

The purified PCR amplicons were self-circularized using Gibson assembly. To favor intra-molecular circularization over inter-molecular ligation, Gibson assembly was performed in a large reaction volume of 1 mL. The following reaction was set up and incubated at 50° C. for 1 hour: 100-1000 ng PCR amplicon, 1× CutSmart buffer (New England Biolabs Inc., Ipswich, MA) and 10 uL 2× Gibson mastermix (New England Biolabs Inc., Ipswich, MA) topped up with water to final 1 mL. Unligated linear templates were removed with 6 U Lambda exonuclease (New England Biolabs Inc., Ipswich, MA) at 37° C. for 30 minutes and inactivated at 65° C. for 20 minutes. Circularized templates were cleaned up with Zymo DCC-5 columns (Zymo Research, Irvine, CA).

A second PCR was set up to generate short fragments to link the locus-of-interest and reverse transcription priming oligo (equivalent to cell ID containing region in a single cell cDNA library) in close proximity, such that they can be sequenced using short-reads Illumina platforms. The primers used in second PCR were two gene-specific primers, each appended with either Illumina P5 or Illumina P7 adapter.

The final sequencing library was cleaned up Zymo DCC-5 columns and quantitated with NEBNext library quantification kit (New England Biolabs Inc., Ipswich, MA). With Illumina platforms, Read 1 sequence includes the reverse transcription priming oligo, while Read 2 provides sequence information on the locus-of-interest, i.e., the mutation.

### Results

Sanger sequencing was used to validate the molecular design for this Illumina sequencing library, using P5 as the Sanger sequencing primer. In FIG. 1C, Sanger sequencing identifies the key elements as per the molecular design for linking distant locus-of-interest to the reverse transcription primer (10× Genomics Cell ID barcode on 3′-end of cDNA).

Preliminary data indicate that the molecular design disclosed herein yields an optimal library that simultaneously provides sequence information on both the locus-of-interest and cDNA tag (barcodes/UMI). Previously, a single-cell RNA-Seq library generated with a droplet or nanowell based platform did not allow researchers to obtain single-cell information on a particular locus-of-interest, and is largely suited for only gene expression profiling. With this molecular workflow, it is now possible to sequence locus-of-interest while preserving the single-cell information provided the single cell cDNA tag.

### Example 2

As depicted in FIG. 4, Alternative B, a plurality of oligonucleotides annealed to the cDNA template in 1× NEB Thermo Pol buffer (New England Biolabs Inc., Ipswich, MA), overnight at 60° C. Annealed oligonucleotides were extended by the addition of Bst DNA polymerase, Large Fragment following the manufacturers protocol (New England Biolabs Inc., Ipswich, MA). The resulting extension products were used as templates for minimal PCR amplification using primers hybridizing to the PCR handle and the truncated Read 1 sequences. The products were then used for self-circularization followed by second round of hemi-nested PCR using second forward and second reverse primers to yield the final sequencing library.

### Example 3

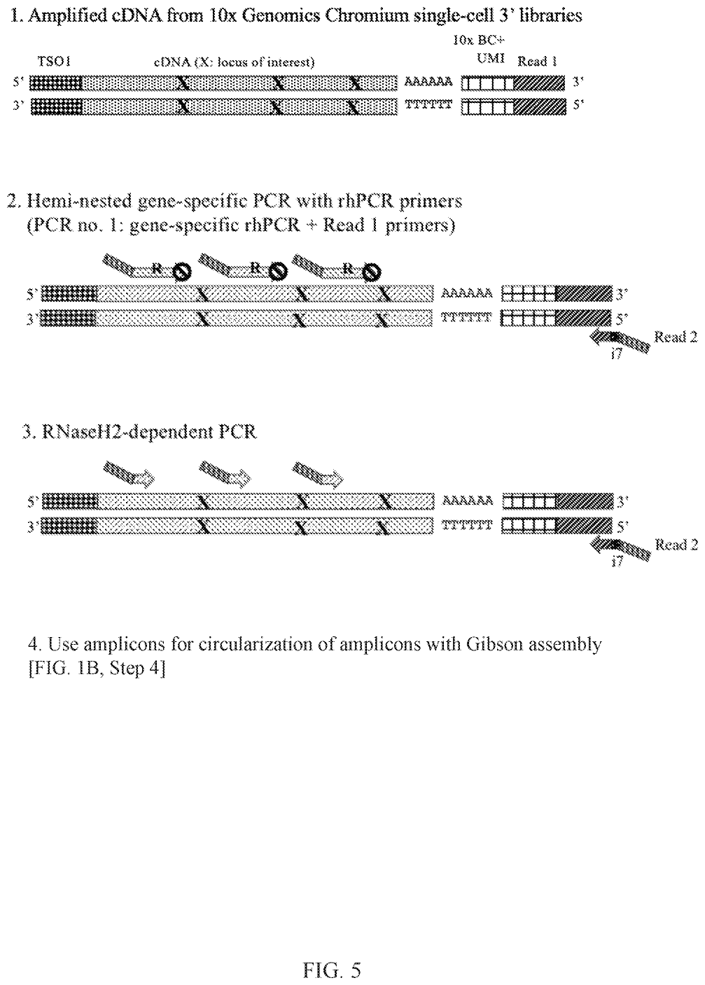

As depicted in FIG. 5, the PCR reaction was performed according to manufacturer's protocol for KAPA HiFi Readymix (Roche, Basel, Switzerland), with the following modifications. The rhPCR primers contain a single ribonucleotide residue and a 3′ blocking moiety. In the annealing step of PCR cycling program, the rhPCR primers annealed to on-targets were activated by cleaving with 1 mU/uL of RNase H2. The products were then used for self-circularization followed by a second round of hemi-nested PCR using the second forward and second reverse primers to yield the final sequencing library.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

TABLE

| SEQ ID | Name | Nucleic Acid Sequence |
|---|---|---|
| NO: 1 | TSO1 | AAGCAGTGGTATCAACGCAGAGTACATrGrGrG |
| NO: 2 | TSO1 | AGAGACAGATTGCGCAATGNNNNNNNNrGrGrG |
| NO: 3 | TSO1 | AAGCAGTGGTATCAACGCAGAGTACATrGrG+G |
| NO: 4 | PCR handle | AAGCAGTGGTATCAACGCAGAGTAC |
| NO: 5 | TSO2 5′ | CTACACGACGCTCTTCCGATCT |
| NO: 6 | TSO2 3′ | TTTCTTATATrGrGrG |
| NO: 7 | first universal | ACACTCTTTCCCTACACGACGCTCTTCCGATCT |
| | sequence | |
| NO: 8 | second universal | ACACGTCTGAACTCC |
| NO: 9 | sequence | GGAGTTCAGACGTGT |
| NO: 10 | cell identification | AAACCTGAGAAACCAT |
| NO: 11 | tag sequence | AAACCTGAGAAACCGC |
| NO: 12 | | AAACCTGAGAAACCTA |
| NO: 13 | | AAACCTGAGAAACGAG |
| NO: 14 | | AAACCTGAGAAACGCC |
| NO: 15 | | AAACCTGAGAAAGTGG |
| NO: 16 | | AAACCTGAGAACAACT |
| NO: 17 | | AAACCTGAGAACAATC |
| NO: 18 | | AAACCTGAGAACTCGG |
| NO: 19 | | AAACCTGAGAACTGTA |
| NO: 20 | (Part of) first | GGAAAGAGTGT |
| NO: 21 | oligonucleotide | GGAGTTCAGACGTGTGCTCTTCCGATCT |
| NO: 22 | First reverse | ACACGTCTGAACTCCAGTCAC |
| | primer 5′ | |
| NO: 23 | | AGATCGGAAGAGCGTCGTGTAGGGAAAGAGTGT |
| NO: 24 | | GTGACTGGAGTTCAGACGTGT |
| NO: 25 | | TTTCTTATATGGG |
| NO: 26 | | AGATCGGAAGAGCACACGTCTGAACTCC |
| NO: 27 | immobilizing | CCTCTCTATGGGCAGTCGGTGAT |
| NO: 28 | oligonucleotide | CCATCTCATCCCTGCGTGTCTCCGACTCAG |
| NO: 29 | | CAAGCAGAAGACGGCATACGAGAT |
| NO: 30 | | AATGATACGGCGACCACCGAGATCTACAC |
| NO: 31 | | GTGACTGGAGTTCAGACGTGTGCTCTTCCGATCT |
| NO: 32 | | AGATCGGAAGAGCACACGTCTGAACTCCAGTCAC |
| NO: 33 | | ATCTCGTATGCCGTCTTCTGCTTG |
| NO: 34 | Q7005 | GTGAATAT |
| NO: 35 | Q7006 | ACAGGCGC |
| NO: 36 | Q7007 | CATAGAGT |
| NO: 37 | Q7008 | TGCGAGAC |
| NO: 38 | Q7015 | TCTCTACT |
| NO: 39 | Q7016 | CTCTCGTC |
| NO: 40 | Q7017 | CCAAGTCT |
| NO: 41 | Q7018 | TTGGACTC |
| NO: 42 | Q7023 | GCAGAATT |
| NO: 43 | Q7024 | ATGAGGCC |
| NO: 44 | Q7025 | ACTAAGAT |
| NO: 45 | Q7026 | GTCGGAGC |
| NO: 46 | Q7027 | AGCCTCAT |
| NO: 47 | Q7028 | GATTCTGC |
| NO: 48 | Q7029 | TCGTAGTG |
| NO: 49 | Q7030 | CTACGACA |
| NO: 50 | Q7035 | ATGGCATG |
| NO: 51 | Q7036 | GCAATGCA |
| NO: 52 | Q7039 | CTTATCGG |
| NO: 53 | Q7040 | TCCGCTAA |
| NO: 54 | Q7041 | GATCTATC |
| NO: 55 | Q7042 | AGCTCGCT |
| NO: 56 | Q7047 | ACACTAAG |
| NO: 57 | Q7048 | GTGTCGGA |

rG: riboguanosine; +G: locked nucleic acid (LNA)-modified guanosine

What is claimed is:

1. A method of generating a tagged DNA amplicon for sequencing, comprising the steps of:
a) annealing a first oligonucleotide to a nucleic acid template comprising a target nucleic acid molecule sequence, a tag sequence and at least a portion of a first universal sequence, wherein the target nucleic acid molecule sequence comprises a locus of interest;
b) performing nucleic acid template-directed nucleic acid extension from the annealed first oligonucleotide to produce a first extension product comprising all or a portion of the target nucleic acid molecule sequence that comprises the locus of interest, the tag sequence and at least a portion of the first universal sequence;
c) circularizing the first extension product to produce a circularized DNA template comprising the locus of interest, the tag sequence, at least a portion of the first universal sequence and at least a portion of a second universal sequence; and
d) performing circularized DNA template-directed nucleic acid amplification to produce a DNA amplicon comprising the locus of interest, the tag sequence, at least a portion of the first universal sequence and at least a portion of the second universal sequence,
thereby generating the tagged DNA amplicon for sequencing.

2. The method of claim 1, wherein:
a) the nucleic acid template comprises a truncated first universal sequence;
b) the circularized DNA template comprises the entire first universal sequence and the entire second universal sequence; and
c) the DNA amplicon comprises the entire first universal sequence and the entire second universal sequence.

3. The method of claim 2, further comprising performing first extension product-directed nucleic acid amplification to amplify the first extension product.

4. The method of claim 3, wherein the first extension product-directed nucleic acid amplification is performed using:
a) the first oligonucleotide; and
b) a first reverse primer comprising at least a portion of the truncated first universal sequence.

5. A method of generating a tagged DNA amplicon for sequencing, comprising the steps of:
a) providing a nucleic acid template comprising a target nucleic acid molecule sequence, a tag sequence and at least a portion of a first universal sequence, wherein the target nucleic acid molecule sequence comprises a locus of interest;
b) performing nucleic acid template-directed nucleic acid amplification to produce a first amplification product by:
i. contacting the nucleic acid template with a reaction mixture comprising a first oligonucleotide and a first reverse primer under conditions in which the first oligonucleotide and the first reverse primer anneal to complementary nucleotide sequences in the nucleic acid template, wherein the first reverse primer comprises at least a portion of the first universal sequence; and
ii. extending each of the first oligonucleotide and the first reverse primer;
c) circularizing the first amplification product to produce a circularized DNA template comprising the locus of interest, the tag sequence, at least a portion of the first universal sequence and at least a portion of a second universal sequence; and
d) performing circularized DNA template-directed nucleic acid amplification to produce a DNA amplicon comprising the locus of interest, the tag sequence, at least a portion of the first universal sequence and at least a portion of the second universal sequence,
thereby generating the tagged DNA amplicon for sequencing.

6. The method of claim 5, wherein:
a) the nucleic acid template comprises a truncated first universal sequence;
b) the circularized DNA template comprises the entire first universal sequence and the entire second universal sequence; and
c) the DNA amplicon comprises the entire first universal sequence and the entire second universal sequence.

7. The method of any one of claims 1-6, wherein the nucleic acid template is a complementary DNA (cDNA) template.

8. The method of claim 7, wherein the cDNA template is obtained by reverse transcribing an RNA from a single cell.

9. The method of claim 8, wherein the RNA is mRNA.

10. The method of any one of claims 1-9, wherein the cDNA template corresponds to a first strand cDNA that comprises, from the 5′ end to the 3′ end, the truncated first universal sequence, the tag sequence and the target nucleic acid molecule sequence.

11. The method of claim 10, wherein the cDNA template comprises, from the 5′ end to the 3′ end, the truncated first universal sequence, the tag sequence, the poly(T) sequence, the target nucleic acid molecule sequence and a first template switching oligo (TSO1) sequence.

12. The method of any one of claims 1-9, wherein the cDNA template corresponds to a second strand cDNA that comprises, from the 5′ end to the 3′ end, the truncated first universal sequence, the tag sequence, the target nucleic acid molecule sequence.

13. The method of claim 12, wherein the cDNA template comprises, from the 5′ end to the 3′ end, the truncated first universal sequence, the tag sequence, a second template switching oligo (TSO2) sequence, the target nucleic acid molecule sequence, the poly(A) sequence and the PCR handle sequence.

14. The method of any one of claims 1-13, wherein the locus of interest comprises a mutation, a polymorphism, an insertion, a deletion, a gene fusion, an edited nucleotide, a modified nucleotide, a transgene or a combination thereof.

15. The method of any one of claims 1-14, wherein the tag sequence comprises a cell identification tag or a unique molecular identifier (UMI) sequence, or a combination thereof.

16. The method of any one of claims 1-15, wherein the first oligonucleotide anneals to a target nucleic acid molecule sequence that is about 0-150 nucleotides 3′ of the locus of interest.

17. The method of any one of claims 1-16, wherein the first oligonucleotide comprises, from the 5′ end to the 3′ end, a second universal sequence and a target nucleic acid molecule-specific sequence.

18. The method of any one of claims 1-17, wherein the first extension product or the first amplification product comprises, from the 5′ end to the 3′ end, all or a portion of the target nucleic acid molecule sequence comprising the locus of interest, the tag sequence and the at least a portion of the truncated first universal sequence.

19. The method of any one of claims 4-18, wherein the first oligonucleotide and the first reverse primer further comprise a second universal sequence.

20. The method of any one of claims 4-19, wherein:
a) the first oligonucleotide or the first reverse primer further comprises, at the 3′ end, an RNA base followed by a blocking domain; and
b) amplifying the first extension product comprises performing an RNase H-dependent PCR.

21. The method of any one of claims 1-20, wherein the method comprises a single circularizing step.

22. The method of any one of claims 1-21, wherein circularizing the first extension product or the first amplification product comprises an intramolecular ligation mediated by Gibson assembly, splint ligation, or a ligation using a thermostable ATP-dependent ligase.

23. The method of claim 22, wherein circularizing the first extension product or the first amplification product comprises an intramolecular ligation mediated by Gibson assembly.

24. The method of any one of claims 1-23, wherein performing circularized DNA template-directed nucleic acid amplification comprises amplifying a portion of the circularized DNA template with a high-fidelity DNA Polymerase.

25. The method of any one of claims 1-24, wherein performing circularized DNA template-directed nucleic acid amplification uses a second forward primer and a second reverse primer.

26. The method of claim 25, wherein the second forward primer or the second reverse primer comprises an oligo dT sequence, a barcoded random nucleotide sequence or a combination thereof.

27. The method of claim 25 or 26, wherein:
a) the second forward primer comprises a sequence that is reversed and complementary to a first immobilizing oligonucleotide sequence; and
b) the second reverse primer comprises a sequence that is reversed and complementary to a second immobilizing oligonucleotide sequence.

28. The method of claim 27, wherein the first and the second immobilizing oligonucleotide sequences comprise:
a)
(SEQ ID NO: 27)
CCTCTCTATGGGCAGTCGGTGAT
and
(SEQ ID NO: 28)
CCATCTCATCCCTGCGTGTCTCCGACTCAG, respectively;
b)
(SEQ ID NO: 28)
CCATCTCATCCCTGCGTGTCTCCGACTCAG
and
(SEQ ID NO: 27)
CCTCTCTATGGGCAGTCGGTGAT, respectively;
or
c)
(SEQ ID NO: 29)
CAAGCAGAAGACGGCATACGAGAT
and
(SEQ ID NO: 30)
AATGATACGGCGACCACCGAGATCTACAC, respectively.

29. The method of any one of claims 25-28, wherein:
a) the second forward primer or the second reverse primer comprises an RNA base followed by a blocking domain at the 3′ end; and
b) performing circularized DNA template-directed nucleic acid amplification comprises performing an RNase H-dependent PCR.

30. The method of any one of claims 1-29, wherein the DNA amplicon comprises, from the 5′ end to the 3′ end, the locus of interest, the second universal sequence, the first universal sequence and the tag sequence.

31. The method of any one of claims 1-30, wherein the DNA amplicon is between about 200 base pairs and about 500 base pairs in length.

32. The method of any one of claims 1-31, wherein the target nucleic acid molecule sequence comprises at least two loci of interest.

33. The method of claim 32, wherein the first oligonucleotide anneals to a target nucleic acid molecule sequence that is 3′ to the loci of interest.

34. The method of claim 32, wherein the method comprises the steps of:
a) annealing a plurality of oligonucleotides to the cDNA template;
b) performing cDNA template-directed nucleic acid extensions from the plurality of annealed oligonucleotides to produce a plurality of first extension products comprising all or a portion of target nucleic acid molecule sequences that comprise one or more loci of interest, the tag sequence and at least a portion of the truncated first universal sequence;
c) circularizing the plurality of first extension products to produce a plurality of circularized DNA templates; and
d) performing circularized DNA template-directed nucleic acid amplifications to produce a plurality of DNA amplicons comprising the one or more loci of interest, the tag sequence, the first universal sequence and the second universal sequence,
thereby generating a plurality of tagged DNA amplicons for sequencing.

35. The method of claim 34, further comprising performing first extension product-directed nucleic acid amplification to amplify the plurality of first extension products.

36. The method of claim 32, wherein the method comprises the steps of:
a) providing the nucleic acid template;
b) performing nucleic acid template-directed nucleic acid amplification to produce a plurality of amplification product by:
i. contacting the nucleic acid template with a reaction mixture comprising a plurality of oligonucleotide primers and a first reverse primer under conditions in which the plurality of oligonucleotide primers and the first reverse primer anneal to complementary nucleotide sequences in the nucleic acid template; and
ii. extending each of the plurality of oligonucleotides and the first reverse primer;
c) circularizing the plurality of first amplification products to produce a plurality of circularized DNA templates; and
d) performing circularized DNA template-directed nucleic acid amplifications to produce a plurality of DNA amplicons comprising the one or more loci of interest, the tag sequence, at least a portion of the first universal sequence and at least a portion of the second universal sequence,
thereby generating a plurality of tagged DNA amplicons for sequencing.

37. The method of claim 36, wherein the plurality of DNA amplicons comprise the first universal sequence and the second universal sequence

38. The method of any one of claims 1-37, wherein the method is used to make a sequencing library.

39. The method of any one of claims 1-38, further comprising sequencing the DNA amplicon.

40. The method of claim 39, wherein the sequencing is performed using a first universal sequence-specific primer, a second universal sequence-specific primer or a combination of the foregoing.

41. The method of claim 40, wherein the sequencing is performed using a primer comprising all or a portion of:
a)
(SEQ ID NO: 7)
ACACTCTTTCCCTACACGACGCTCTTCCGATCT;
b)
(SEQ ID NO: 31)
GTGACTGGAGTTCAGACGTGTGCTCTTCCGATCT;
or
c)
(SEQ ID NO: 32)
AGATCGGAAGAGCACACGTCTGAACTCCAGTCAC,
or a combination of the foregoing.

42. A method of generating a tagged DNA amplicon for sequencing, comprising the steps of:
a) providing a nucleic acid template comprising, from the 5′ end to the 3′ end, a truncated first universal sequence, a tag sequence, a target nucleic acid molecule sequence and a first template switching oligo (TSO1) sequence, wherein the target nucleic acid molecule sequence comprises a locus of interest;
b) performing nucleic acid template-directed nucleic acid amplification to produce a first amplification product by:
i. contacting the nucleic acid template with a reaction mixture comprising a first oligonucleotide and a first reverse primer under conditions in which the first oligonucleotide and the first reverse primer anneal to complementary nucleotide sequences in the nucleic acid template, wherein:
1) the first oligonucleotide comprises, from the 5′ end to the 3′ end, a second universal sequence and a target nucleic acid molecule-specific sequence; and
2) the first reverse primer comprises, from the 5′ end to the 3′ end, a second universal sequence, [i7], the missing first universal sequence and at least a portion of the truncated first universal sequence; and
ii. extending each of the first oligonucleotide and the first reverse primer;
c) circularizing the first amplification product to produce a circularized DNA template comprising the second universal sequence, the locus of interest, the tag sequence, the first universal sequence and [i7]; and
d) performing circularized DNA template-directed nucleic acid amplification to produce a DNA amplicon comprising, from 5′ end to the 3′ end, the Illumina P5 sequence, the locus of interest, the second universal sequence, [i7], the first universal sequence, the tag sequence and the Illumina P7 sequence,
thereby generating the tagged DNA amplicon for sequencing.

43. A method of generating a tagged DNA amplicon for sequencing, comprising the steps of:
a) providing a nucleic acid template comprising, from the 5′ end to the 3′ end, a truncated first universal sequence, a tag sequence, a second template switching oligo (TSO2) sequence and a target nucleic acid molecule sequence, wherein the target nucleic acid molecule sequence comprises a locus of interest;
b) performing nucleic acid template-directed nucleic acid amplification to produce a first amplification product by:
i. contacting the nucleic acid template with a reaction mixture comprising a first oligonucleotide and a first reverse primer under conditions in which the first oligonucleotide and the first reverse primer anneal to complementary nucleotide sequences in the nucleic acid template, wherein:
1) the first oligonucleotide comprises, from the 5′ end to the 3′ end, a second universal sequence and a target nucleic acid molecule-specific sequence; and
2) the first reverse primer comprises, from the 5′ end to the 3′ end, a second universal sequence, [i7], the missing first universal sequence and at least a portion of the truncated first universal sequence; and
ii. extending each of the first oligonucleotide and the first reverse primer;
c) circularizing the first amplification product to produce a circularized DNA template comprising the second universal sequence, the locus of interest, the TSO2 sequence, the tag sequence, the first universal sequence and [i7]; and
d) performing circularized DNA template-directed nucleic acid amplification to produce a DNA amplicon comprising, from 5′ end to the 3′ end, the Illumina P5 sequence, the locus of interest, the second universal sequence, [i7], the first universal sequence, the TSO2 sequence, the tag sequence and the Illumina P7 sequence,
thereby generating the tagged DNA amplicon for sequencing.

44. A tagged DNA amplicon for sequencing, comprising, from the 5′ end to the 3′ end, a locus of interest, at least a portion of a second universal sequence, at least a portion of a first universal sequence and a tag sequence.

45. The tagged DNA amplicon of claim 44, comprising, from the 5′ end to the 3′ end, a locus of interest, the second universal sequence, the first universal sequence and the tag sequence.

46. The tagged DNA amplicon of claim 45, comprising, from the 5′ end to the 3′ end, an Illumina P5 sequence, the locus of interest, the second universal sequence, the first universal sequence, the tag sequence and the Illumina P7 sequence.

47. The tagged DNA amplicon of claim 46, comprising, from the 5′ end to the 3′ end, an Illumina P5 sequence, the locus of interest, the second universal sequence, [i7], the first universal sequence, the tag sequence and the Illumina P7 sequence.

48. The tagged DNA amplicon of claim 47 comprising, from the 5′ end to the 3′ end, an Illumina P5 sequence, the locus of interest, the second universal sequence, [i7], the first universal sequence, the tag sequence, a poly(T) sequence and the Illumina P7 sequence.

49. The tagged DNA amplicon of claim 47 comprising, from the 5′ end to the 3′ end, an Illumina P5 sequence, the locus of interest, the second universal sequence, [i7], a second template switching oligo (TSO2) sequence and the Illumina P7 sequence.


SEQUENCE LISTING (57 sequences)

SEQ ID NO: 1 - DNA, 30 bp, Artificial Sequence
  Synthetic oligonucleotide
  modified_base (28)...(30): n is riboguanosine
aagcagtggtatcaacgcagagtacatnnn

SEQ ID NO: 2 - DNA, 30 bp, Artificial Sequence
  Synthetic oligonucleotide
  misc_feature (20)...(27): n is a, c, g, or t
  misc_feature (28)...(30): n is riboguanosine
agagacagattgcgcaatgnnnnnnnnnnn

SEQ ID NO: 3 - DNA, 30 bp, Artificial Sequence; identical to SEQ ID NO: 1
  Synthetic oligonucleotide
  misc_feature (28)...(29): n is riboguanosine
  misc_feature (30)...(30): n is locked nucleic acid (LNA)-modified guanosine (+G)

SEQ ID NO: 4 - DNA, 25 bp, Artificial Sequence
  Synthetic oligonucleotide
aagcagtggtatcaacgcagagtac

SEQ ID NO: 5 - DNA, 22 bp, Artificial Sequence
  Synthetic oligonucleotide
ctacacgacgctcttccgatct

SEQ ID NO: 6 - DNA, 13 bp, Artificial Sequence
  Synthetic oligonucleotide
  misc_feature (11)...(13): n is riboguanosine
tttcttatatnnn

SEQ ID NO: 7 - DNA, 33 bp, Artificial Sequence
  Synthetic oligonucleotide
acactctttccctacacgacgctcttccgatct

SEQ ID NO: 8 - DNA, 15 bp, Artificial Sequence
  Synthetic oligonucleotide
acacgtctgaactcc

SEQ ID NO: 9 - DNA, 15 bp, Artificial Sequence
  Synthetic oligonucleotide
ggagttcagacgtgt

SEQ ID NO: 10 - DNA, 16 bp, Artificial Sequence
  Synthetic oligonucleotide
aaacctgagaaaccat

SEQ ID NO: 11 - DNA, 16 bp, Artificial Sequence
  Synthetic oligonucleotide
aaacctgagaaaccgc

SEQ ID NO: 12 - DNA, 16 bp, Artificial Sequence
  Synthetic oligonucleotide
aaacctgagaaaccta

SEQ ID NO: 13 - DNA, 16 bp, Artificial Sequence
  Synthetic oligonucleotide
aaacctgagaaacgag

SEQ ID NO: 14 - DNA, 16 bp, Artificial Sequence
  Synthetic oligonucleotide
aaacctgagaaacgcc

SEQ ID NO: 15 - DNA, 16 bp, Artificial Sequence
  Synthetic oligonucleotide
aaacctgagaaagtgg

SEQ ID NO: 16 - DNA, 16 bp, Artificial Sequence
  Synthetic oligonucleotide
aaacctgagaacaact

SEQ ID NO: 17 - DNA, 16 bp, Artificial Sequence
  Synthetic oligonucleotide
aaacctgagaacaatc

SEQ ID NO: 18 - DNA, 16 bp, Artificial Sequence
  Synthetic oligonucleotide
aaacctgagaactcgg

SEQ ID NO: 19 - DNA, 16 bp, Artificial Sequence
  Synthetic oligonucleotide
aaacctgagaactgta

SEQ ID NO: 20 - DNA, 11 bp, Artificial Sequence
  Synthetic oligonucleotide
ggaaagagtgt

SEQ ID NO: 21 - DNA, 28 bp, Artificial Sequence
  Synthetic oligonucleotide
ggagttcagacgtgtgctcttccgatct

SEQ ID NO: 22 - DNA, 21 bp, Artificial Sequence
  Synthetic oligonucleotide
acacgtctgaactccagtcac

SEQ ID NO: 23 - DNA, 33 bp, Artificial Sequence
  Synthetic oligonucleotide
agatcggaagagcgtcgtgtagggaaagagtgt

SEQ ID NO: 24 - DNA, 21 bp, Artificial Sequence
  Synthetic oligonucleotide
gtgactggagttcagacgtgt

SEQ ID NO: 25 - DNA, 13 bp, Artificial Sequence
  Synthetic oligonucleotide
tttcttatatggg

SEQ ID NO: 26 - DNA, 28 bp, Artificial Sequence
  Synthetic oligonucleotide
agatcggaagagcacacgtctgaactcc

SEQ ID NO: 27 - DNA, 23 bp, Artificial Sequence
  Synthetic oligonucleotide
cctctctatgggcagtcggtgat

SEQ ID NO: 28 - DNA, 30 bp, Artificial Sequence
  Synthetic oligonucleotide
ccatctcatccctgcgtgtctccgactcag

SEQ ID NO: 29 - DNA, 24 bp, Artificial Sequence
  Synthetic oligonucleotide
caagcagaagacggcatacgagat

SEQ ID NO: 30 - DNA, 29 bp, Artificial Sequence
  Synthetic oligonucleotide
aatgatacggcgaccaccgagatctacac

SEQ ID NO: 31 - DNA, 34 bp, Artificial Sequence
  Synthetic oligonucleotide
gtgactggagttcagacgtgtgctcttccgatct

SEQ ID NO: 32 - DNA, 34 bp, Artificial Sequence
  Synthetic oligonucleotide
agatcggaagagcacacgtctgaactccagtcac

SEQ ID NO: 33 - DNA, 24 bp, Artificial Sequence
  Synthetic oligonucleotide
atctcgtatgccgtcttctgcttg

SEQ ID NO: 34 - DNA, 8 bp, Artificial Sequence
  Synthetic oligonucleotide
gtgaatat

SEQ ID NO: 35 - DNA, 8 bp, Artificial Sequence
  Synthetic oligonucleotide
acaggcgc

SEQ ID NO: 36 - DNA, 8 bp, Artificial Sequence
  Synthetic oligonucleotide
catagagt

SEQ ID NO: 37 - DNA, 8 bp, Artificial Sequence
  Synthetic oligonucleotide
tgcgagac

SEQ ID NO: 38 - DNA, 8 bp, Artificial Sequence
  Synthetic oligonucleotide
tctctact

SEQ ID NO: 39 - DNA, 8 bp, Artificial Sequence
  Synthetic oligonucleotide
ctctcgtc

SEQ ID NO: 40 - DNA, 8 bp, Artificial Sequence
  Synthetic oligonucleotide
ccaagtct

SEQ ID NO: 41 - DNA, 8 bp, Artificial Sequence
  Synthetic oligonucleotide
ttggactc

SEQ ID NO: 42 - DNA, 8 bp, Artificial Sequence
  Synthetic oligonucleotide
gcagaatt

SEQ ID NO: 43 - DNA, 8 bp, Artificial Sequence
  Synthetic oligonucleotide
atgaggcc

SEQ ID NO: 44 - DNA, 8 bp, Artificial Sequence
  Synthetic oligonucleotide
actaagat

SEQ ID NO: 45 - DNA, 8 bp, Artificial Sequence
  Synthetic oligonucleotide
gtcggagc

SEQ ID NO: 46 - DNA, 8 bp, Artificial Sequence
  Synthetic oligonucleotide
agcctcat

SEQ ID NO: 47 - DNA, 8 bp, Artificial Sequence
  Synthetic oligonucleotide
gattctgc

SEQ ID NO: 48 - DNA, 8 bp, Artificial Sequence
  Synthetic oligonucleotide
tcgtagtg

SEQ ID NO: 49 - DNA, 8 bp, Artificial Sequence
  Synthetic oligonucleotide
ctacgaca

SEQ ID NO: 50 - DNA, 8 bp, Artificial Sequence
  Synthetic oligonucleotide
atggcatg

SEQ ID NO: 51 - DNA, 8 bp, Artificial Sequence
  Synthetic oligonucleotide
gcaatgca

SEQ ID NO: 52 - DNA, 8 bp, Artificial Sequence
  Synthetic oligonucleotide
cttatcgg

SEQ ID NO: 53 - DNA, 8 bp, Artificial Sequence
  Synthetic oligonucleotide
tccgctaa

SEQ ID NO: 54 - DNA, 8 bp, Artificial Sequence
  Synthetic oligonucleotide
gatctatc

SEQ ID NO: 55 - DNA, 8 bp, Artificial Sequence
  Synthetic oligonucleotide
agctcgct

SEQ ID NO: 56 - DNA, 8 bp, Artificial Sequence
  Synthetic oligonucleotide
acactaag

SEQ ID NO: 57 - DNA, 8 bp, Artificial Sequence
  Synthetic oligonucleotide
gtgtcgga